(19) United States
(12) Patent Application Publication
(10) Pub. No.: US 2022/0133722 A1
(43) Pub. Date: May 5, 2022

(54) PHARMACEUTICAL COMPOSITIONS

(71) Applicant: QUE ONCOLOGY PTY LTD, Melbourne, Victoria (AU)

(72) Inventors: Robert Crombie, Melbourne (AU); Ardian Latifi, Melbourne (AU)

(21) Appl. No.: 17/430,983

(22) PCT Filed: Feb. 14, 2020
(86) PCT No.: PCT/AU2020/050125

§ 371 Date: Aug. 13, 2021

(30) Foreign Application Priority Data
Feb. 15, 2019 (AU) PCT/AU2019/050122

(51) Int. Cl. A61K 31/506; A61J 1/03

(52) U.S. Cl. CPC A61K 31/506; A61J 1/035

(57) ABSTRACT

The present invention relates to pharmaceutical compositions for the treatment or prevention of symptoms associated with endocrine disturbances, such as hot flashes. The pharmaceutical compositions comprise a tricyclic amino containing compound of formula (I), such as MSX-122 (also known as “Q-122”) (N,N′-(1,4-phenylenebis(methylene))bis(pyrimidin-2-amine)), and one or more pharmaceutically acceptable excipients. The pharmaceutical compositions may be given in certain doses and/or dosing regimens, preferably twice daily.

### CROSS-REFERENCE TO EARLIER APPLICATION(S)

This application claims priority from PCT application PCT/AU2019/050122, the entire contents are hereby incorporated by reference in their entirety.

### FIELD OF THE INVENTION

The present invention relates to pharmaceutical compositions for the treatment or prevention of symptoms associated with endocrine disturbances, such as hot flashes. The pharmaceutical compositions comprise a compound of formula (I) and one or more pharmaceutically acceptable excipients. The pharmaceutical compositions may be given in certain doses and/or dosing regimens.

### BACKGROUND OF THE INVENTION

The majority of women experience hot flashes of some kind during the years immediately before and after menopause. A hot flash is characterized by a sudden, intense, hot feeling on the face and upper body. Often the hot flash can be preceded or accompanied by a rapid heartbeat and sweating, nausea, dizziness, anxiety, headache, weakness, or a feeling of suffocation. Some women experience a general, overall uneasy feeling just before the hot flash. A hot flash is generally followed by a flush, leaving the sufferer reddened and perspiring.

High intensity hot flashes can result in the sufferer becoming soaked in perspiration. Lower intensity flashes merely produce a moist upper lip. A chill often precedes the flash, but can also occur at the conclusion of the flash. When hot flashes occur during the night, sleep is likely also adversely affected, resulting in poor concentration, memory problems, irritability and exhaustion during the day.

Hot flashes are considered to be the result of hormonal changes associated with menopause (particularly due to a drop in the level of estrogen), but men can also have hot flashes if their levels of testosterone drop suddenly and dramatically. In addition, hot flashes can also be influenced by lifestyle and medications. For example, both men and women can suffer from hot flashes as a side effect of cancer therapy. Certain drugs such as Tamoxifen (Nolvadex), which is used to treat breast cancer, as well as Lupron (Leuprolide) and Zoladex (Goserelin), which are employed in the therapy of prostate cancer, can lead to heat sensations. Bilateral orchiectomy for prostate cancer or testicular cancer also affects the hormone system so that patients can subsequently suffer from hot flashes.

Symptoms that mimic hot flashes can occur in both men and women who have a tumour of the hypothalamus or pituitary gland, as well as with those who have suffered from certain serious infections, such as tuberculosis or HIV, those with alcoholism or those who suffer from thyroid disorders. Symptoms that are similar to hot flashes also can be a side effect of the food additive monosodium glutamate (MSG), or of certain medications, particularly nitroglycerin, nifedipine, niacin, vancomycin and calcitonin.

Hormone Replacement Therapy (HRT) is believed to be one of the most effective treatments available to reduce the onset of hot flashes. However, HRT has been associated with increased risk of heart disease as well as certain kinds of cancers, and therefore HRT is often not advised in patients undergoing treatment for cancer.

Compounds of formula (I), as defined below, have been shown to be effective in the treatment of hot flashes in patients undergoing endocrine therapy. There is a need for pharmaceutical compositions comprising effective amounts of compounds of formula (I) to prevent or treat symptoms associated with endocrine disturbances, such as hot flashes. There is also a need for dosage regimens for these pharmaceutical compositions that are effective in preventing or treating these symptoms.

### SUMMARY OF THE INVENTION

In a first aspect, the present invention relates to a method of treating or preventing a symptom associated with an endocrine disturbance in a subject in need thereof, comprising administering to the subject an effective amount of a compound of formula (I):

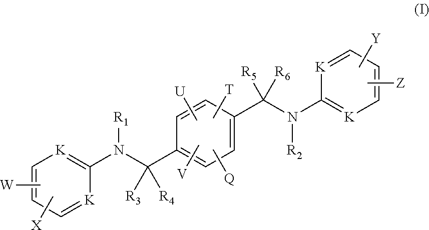

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients.

Q, T, U, and V may be independently selected from H or methyl. Q, T, U, and V may be H.

W, X, Y and Z may be independently selected from H, R, F, Cl, OH, OR or CO₂H. W, X, Y and Z may be independently selected from H, R, F, Cl, OR or CO₂H. W, X, Y and Z may be independently selected from H, R, F, Cl or OR.

R may be a straight chain alkyl group. R may be a branched alkyl group. R may be a C₁-C₁₀ straight chain or branched alkyl group. R may be a C₁-C₅ straight chain or branched alkyl group. R may be a C₁-C₃ straight chain or branched alkyl group. R may be methyl or ethyl.

R₁ and R₂ may be H.

R₃, R₄, R₅ and R₆ may be H.

The compound may be a compound of formula (Ia):

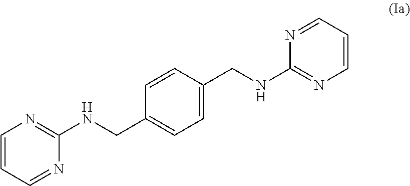 [SMILES: c1cnc(NCc2ccc(CNc3ncccn3)cc2)nc1]

or a pharmaceutically acceptable salt or prodrug thereof.

The effective amount may be from about 50 mg to about 400 mg per dose. The effective amount may be from about 60mg to about 400mg, about 60 mg to about 350 mg, about 70 mg to about 300 mg, about 80 mg to about 250 mg, or about 90 mg to about 200 mg. The effective amount may be about 100 mg per dose. The effective amount may be about 200 mg per dose.

The invention also provides a pharmaceutical composition comprising a compound of formula (I) or a pharmaceutically acceptable salt or prodrug thereof, and one or more pharmaceutically acceptable excipients. The pharmaceutical composition is suitable for use in the methods of the invention.

The pharmaceutical composition may be formulated for oral administration. The pharmaceutical composition may be suitable for, or adapted for, administration once a day. The pharmaceutical composition may be suitable for, or adapted for, administration twice a day. The pharmaceutical composition may be suitable for administration with a consumable. The consumable may be a high-fat consumable. The high-fat consumable may be a high-fat food or meal, or an edible fat or oil.

The invention also provides a unit dosage form comprising a compound of formula (I) or a pharmaceutically acceptable salt or prodrug thereof in an amount sufficient for, suitable for, or adapted for, twice daily administration to a subject in need thereof. The unit dosage form may therefore comprise the compound of formula (I) or a pharmaceutically acceptable salt or prodrug thereof in any effective amount described herein.

The invention also provides a kit comprising in separate parts, a first unit dosage form of the invention and a second unit dosage form of the invention

The first and second unit dosage forms of the kits of the invention may comprise the same amount of the compound of formula (I) or a pharmaceutically acceptable salt or prodrug thereof.

The first and second dosage unit may each separately comprise between about 50 mg and about 400 mg of compound (I) or (Ia) as described herein. More preferably, the first and second dosage unit each separately comprise about 200 mg of compound (I) or (Ia) as described herein. Most preferably, the first and second dosage unit each separately comprise about 100 mg of compound (I) or (Ia) as described herein.

In a second aspect, the present invention relates to a method of treating or preventing a symptom associated with an endocrine disturbance in a subject in need thereof, the method comprising administering to the subject an effective amount of a pharmaceutical composition of the invention or a unit dosage form of the invention.

The present invention also relates to a method of treating or preventing hot flashes in a subject, the method comprising administering an effective amount of a compound or a pharmaceutically acceptable salt or prodrug thereof, or pharmaceutical composition as described herein to an individual in combination with, or at the same time as, consumption of a high-fat consumable. The high-fat consumable may be a high-fat food or meal, or an edible fat or oil.

The present invention also relates to a method of treating or preventing hot flashes in a subject, the method comprising administering a compound of formula (I), (Ia) or a pharmaceutical composition comprising formula (I) or (Ia) as described herein in an amount and at an interval of 100 mg of compound of formula (I) or (Ia) twice every 24 hours (e.g. per day). The 100 mg may be administered once every 12 hours.

The present invention relates to a method of treating or preventing a symptom associated with an endocrine disturbance in a subject in need thereof, the method comprising administering to the subject a pharmaceutical composition as described herein, wherein the pharmaceutical composition comprises a compound of formula (I) or (Ia) in an amount sufficient to provide a C_{trough} of about 150 to about 350 ng/mL when the composition is orally administered to a subject. The pharmaceutical composition may provide a mean C_{trough} of about 230 ng/mL when the composition is orally administered to a subject. The pharmaceutical composition may provide a C_{trough} of greater than 130 ng/mL when the composition is orally administered to a subject. The pharmaceutical composition may be administered in an amount and at an interval of 100 mg of compound of formula (I) or (Ia) twice every 24 hours (e.g. per day). The 100 mg may be administered once every 12 hours. The pharmaceutical composition may be administered in combination with, or at the same time as, a consumable. The consumable may be a high-fat consumable. The high-fat consumable may be a high-fat food or meal, or an edible fat or oil.

The present invention also relates to the use of a compound of formula (I) or a pharmaceutically acceptable salt or prodrug thereof in the manufacture of a medicament for treating or preventing a symptom associated with an endocrine disturbance. In preferred embodiments, the medicament is formulated for twice daily administration of the compound of formula (I) or a pharmaceutically acceptable salt or prodrug thereof to a subject in need thereof.

The present invention also relates to the use of a an effective amount of a compound of formula (I) or a pharmaceutically acceptable salt or prodrug thereof for the treatment or prevention of a symptom associated with an endocrine disturbance in a subject. In preferred embodiments, the effective amount is sufficient for, suitable for, or adapted for, twice daily administration to a subject in need thereof.

The present invention relates to a compound of formula (I), (Ia) or a pharmaceutically acceptable salt or prodrug thereof, or a pharmaceutical composition comprising a compound of formula (I) or (Ia) or a pharmaceutically acceptable salt or prodrug thereof for use in the treatment or prevention of a symptom associated with an endocrine disturbance in a subject. The treatment or prevention may comprise twice daily administration of the compound of formula (I), (Ia) or a pharmaceutically acceptable salt or prodrug thereof.

The pharmaceutical composition may be administered with a consumable. The consumable may be a high-fat food or meal. The compound of formula (I) may be administered to the subject in an amount of between about 50 mg and about 400 mg per dose. The amount may be between about 60 mg and about 350 mg, between about 70 mg and about 300 mg, between about 80 mg and about 250 mg, or between about 90 mg and about 200 mg. The amount may be about 100 mg per dose. The amount may be about 200 mg per dose. The compound of formula (I) may be a compound of formula (Ia).

In any aspect, the symptom may be a hot flash.

In a third aspect, the present invention relates to a pharmaceutical composition according to the first aspect, wherein the pharmaceutical composition comprises a compound of formula (I) or a pharmaceutically acceptable salt or prodrug thereof in an amount sufficient to provide a C_{trough} of about 150 to about 350 ng/mL when the composition is orally administered to a subject.

The pharmaceutical composition may provide a mean C_{trough} of about 230 ng/mL when the composition is orally administered to a subject.

The pharmaceutical composition may provide a C_{trough} of greater than 130 ng/mL when the composition is orally administered to a subject.

The compound of formula (I) may be a compound of formula (Ia).

Administration of a subject with a compound of formula (I), (Ia) or a pharmaceutical composition comprising a compound of formula (I) or (Ia) as described herein may be given in an amount and/or at sufficient interval to achieve and/or maintain a certain quantity of a compound of formula (I) or (Ia) as described herein per volume of serum, using, for example, an assay as described herein. For example, a compound of formula (I), (Ia) or a pharmaceutically acceptable salt or prodrug thereof, or a pharmaceutical composition comprising a compound of formula (I) or (Ia) or a pharmaceutically acceptable salt or prodrug thereof, may be administered to achieve a C_{trough} of about 150 to about 350 ng/m L.

As used herein, except where the context requires otherwise, the term “comprise” and variations of the term, such as “comprising”, “comprises” and “comprised”, are not intended to exclude further additives, components, integers or steps.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Mean (+SD) profiles of fed versus fasted 200 mg doses of a compound of formula (Ia) (“Q-122”) linear-linear (blue circles=fasted, red triangles=fed).

FIG. 2. Mean (+SD) profiles of fed versus fasted 200 mg doses of a compound of formula (Ia) (“Q-122”) log-linear (blue circles=fasted, red triangles=fed).

FIG. 3. Overlay profiles of 100 mg BID dosing of a compound of formula (Ia) (“Q-122”) linear-linear.

FIG. 4. Overlay profiles of 200 mg QD dosing of a compound of formula (Ia) (“Q-122”) linear-linear.

FIG. 5. Trough levels from 100 mg BID dosing of a compound of formula (Ia) (“Q-122”).

FIG. 6. Trough levels from 200 mg QD dosing of a compound of formula (Ia) (“Q-122”).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Therefore, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

The present inventors have found that trough concentrations with BID (i.e. twice-daily) dosing are approximately double those of QD (i.e. once daily) dosing. Surprisingly, administering the same dosage according to a twice daily regimen instead of once-a-day improves the therapeutic profile of the dosage regimen due to increased C_{trough} without proportional increases in Cₘₐₓ. This is particularly advantageous in oncology patients, who may experience decreased exposure to the medication as a result of their disease and the fact that they are not always able to take the medication with food. Twice-daily dosing therefore provides for greater certainty of efficacy in a greater number of these subjects.

The present inventors have also found that a pharmaceutical composition of the present invention, when administered with a high-fat meal, led to a clinically meaningful compound of formula (Ia) exposure increase. Compared to the fasted state, a high-fat meal increased mean Cₘₐₓ and mean AUC_{0-inf} in healthy subjects by about 60% and 45%, respectively. In addition, the pharmacokinetics of the composition is such that the compound of formula (I) accumulates and therefore maintains therapeutic plasma levels, while not significantly affecting the maximum plasma concentration, thereby avoiding potential problems such as overdosing and adverse toxicological effects that may occur with higher drug concentrations.

Compounds

Compounds are generally described herein using standard nomenclature. For compounds having asymmetric centres, it will be understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z and E forms, with all isomeric forms of the compounds being included in the present invention unless otherwise specified. Where a compound exists in various tautomeric forms, a recited compound is not limited to any one specific tautomer, but rather is intended to encompass all tautomeric forms.

Compounds according to the formula provided herein, which have one or more stereogenic centres, may have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single stereoisomers (optically active forms) can be obtained by asymmetric synthesis, synthesis from optically pure precursors, biosynthesis or by resolution of the racemates, for example, enzymatic resolution or resolution by conventional methods such as crystallization in the presence of a resolving agent, or chromatography, using, for example, a chiral HPLC column.

Certain compounds are described herein using a general formula that includes variables such as R, R₁, R₂, W, X, Y, Z, etc. Unless otherwise specified, each variable within such a formula is defined independently of any other variable, and any variable that occurs more than one time in a formula is defined independently at each occurrence. Therefore, for example, if a group is shown to be substituted with 1 or 2 R*, the group may be substituted with up to two R* groups and R* at each occurrence is selected independently from the definition of R*. Also, combinations of substituents and/or variables are permissible only if such combinations result in stable compounds, i.e., compounds that can be isolated, characterized and tested for biological activity.

Compounds of formula (I) have the following general structure:

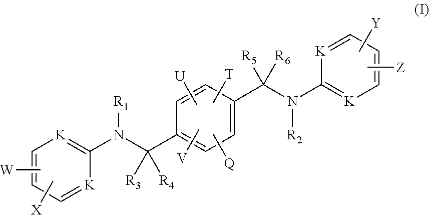

wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R.

As used herein, the term “alkyl” refers to a saturated, straight-chain or branched hydrocarbon group. The term “C₁-C₁₀ alkyl” group refers to an alkyl group having from 1 to 10 carbon atoms and encompasses alkyl groups having 1, 2, 3, 4, 5, 6, 7, 8, 9 or 10 carbon atoms and any sub group thereof, including C₁-C₃ alkyl groups. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl.

Compounds of formula (I), for use in the pharmaceutical composition, methods, uses, kits and unit dosage forms of the present invention, include those where at least three K's are N. All four K's may be N.

Q, T, U, and V may be independently selected from H or methyl. Q, T, U, and V may be H.

R may be a straight chain alkyl group. R may be a branched alkyl group. R may be a C₁-C₁₀ straight chain or branched alkyl group. R may be a C₁-C₅ straight chain or branched alkyl group. R may be a C₁-C₃ straight chain or branched alkyl group. R may be methyl or ethyl.

W, X, Y and Z may be independently selected from H, R, F, Cl, OH, OR or CO₂H. W, X, Y and Z may be independently selected from H, R, F, Cl, OR or CO₂H. W, X, Y and Z may be independently selected from H, R, F, Cl or OR.

R₁ and R₂ may be H.

R₃, R₄, R₅ and R₆ may be H.

The compound of formula (I) may be a compound of formula (Ia):

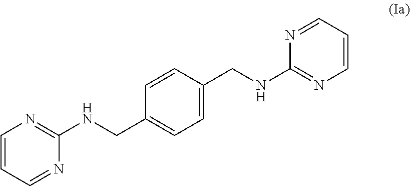 [SMILES: c1cnc(NCc2ccc(CNc3ncccn3)cc2)nc1]

or a pharmaceutically acceptable salt or prodrug thereof.

As used herein, wording defining the limits of a range of length such as, for example, “from 1 to 5” or “1-5” means any integer from 1 to 5, i.e. 1, 2, 3, 4 and 5. In other words, any range defined by two integers explicitly mentioned is meant to comprise and disclose any integer defining said limits and any integer comprised in said range.

A “pharmaceutically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. In particular, pharmaceutically acceptable salts in accordance with the present invention are those that do not adversely affect the therapeutic activity of the compound. Such salts include mineral and organic acid salts of basic residues such as amines, as well as alkali or organic salts of acidic residues such as carboxylic acids.

Suitable pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzenesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic (such as acetic, HOOC—(CH₂)ₙ—COOH where n is any integer from 0 to 6, i.e. 0, 1, 2, 3, 4, 5 or 6), and the like. Similarly, pharmaceutically acceptable cations include, but are not limited to sodium, potassium, calcium, aluminum, lithium and ammonium. A person skilled in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent (such as ether, ethyl acetate, ethanol, isopropanol or acetonitrile), or in a mixture of the two.

It will be apparent that each compound of formula (I) may, but need not, be present as a hydrate, solvate or non-covalent complex. In addition, the various crystal forms and polymorphs are within the scope of the present invention. The compound of formula (I) may also be present as an N-oxide.

Prodrugs of the compounds of formula (I) are also provided herein.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy or amine groups are bonded to any group that, when administered to a mammalian subject, cleaves to form a free hydroxy, carboxy or amino group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Compounds of formula (I) can be prepared using the synthetic methods described in WO 2006/074426 and WO 2006/074428.

Pharmaceutical Compositions

In one aspect, the present invention relates to a pharmaceutical composition comprising a compound of formula (I) or a pharmaceutically acceptable salt or prodrug thereof and one or more pharmaceutically acceptable excipients. The pharmaceutical composition may be provided in the form of a unit dosage form comprising an effective amount of the compound of formula (I) or a pharmaceutically acceptable salt or prodrug thereof and the one or more pharmaceutically acceptable excipients. The effective amount is preferably sufficient for, suitable for, or adapted for, twice daily administration to a subject in need thereof.

The effective amount may be between about 50 mg and about 400 mg per dose. The effective amount may be between about 60 mg and about 350 mg, between about 70 mg and about 300 mg, between about 80 mg and about 250 mg, or between about 90 mg and about 200 mg. The effective amount may be about 100 mg per dose. The effective amount may be about 200 mg per dose. As used herein, the term “effective amount” is intended to refer to an amount of the compound of formula (I) that is effective in treating or preventing a symptom associated with an endocrine disturbance in a subject.

The pharmaceutical composition may be formulated for oral administration. The pharmaceutical composition may be suitable for administration once a day. The term “once a day” as used herein is intended to refer to once in a 24-hour period.

The pharmaceutical composition is preferably suitable for administration twice a day. The term “twice a day” as used herein is intended to refer to administration twice in a 24-hour period. The pharmaceutical composition may be administered in the morning (e.g. with the morning meal) and in the evening (e.g. with the evening meal). The pharmaceutical composition may be administered once every 12 hours.

The pharmaceutical composition may be suitable for administration with a high-fat consumable. The high-fat consumable may be a high-fat food or meal, or an edible fat or oil, such as fish oil.

As used herein, a high-fat consumable is intended to refer to a food, meal or other edible composition having approximately 800 to 1000 calories and where approximately 50 percent of total caloric content of the meal comes from the fat contained therein. The consumable may derive approximately 150, 250, and 500-600 calories from protein, carbohydrate, and fat, respectively. An example meal is two eggs fried in butter, two strips of bacon, two slices of toast with butter, four ounces of hash brown potatoes and eight ounces of whole milk.

The pharmaceutical composition may be suitable for administration in any pharmaceutically acceptable manner. A compound of formula (I) can be combined as the active ingredient in intimate admixture with a pharmaceutically acceptable excipient according to conventional pharmaceutical compounding techniques. The pharmaceutically acceptable excipient may take a wide variety of forms depending on the form of preparation desired for administration, e.g. oral, parenteral (including intravenous, subcutaneous, intrathecal, and intramuscular), transdermal, and topical. The pharmaceutical composition may be formulated for oral administration. In preparing the compositions for oral administration, any of the usual pharmaceutical excipients may be employed. These include, for example, water, glycols, oils, alcohols, flavoring agents, preservatives, colouring agents and the like in the case of oral liquid preparations such as suspensions, elixirs and solutions; or aerosols; or excipients such as starches, sugars, microcrystalline cellulose, diluents, granulating agents, lubricants, binders, disintegrating agents and the like in the case of oral solid preparations such as powders, capsules, caplets, and tablets. Solid oral preparations are generally preferred over liquid ones. Because of their ease of administration, tablets and capsules represent the most advantageous oral dosage unit forms, in which case solid pharmaceutical pharmaceutically acceptable excipients are obviously employed. If desired, tablets may be coated by standard aqueous or non-aqueous techniques.

The composition may be in the form of a discrete unit dosage form, such as a tablet or a capsule. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavouring agent such as peppermint, methyl salicylate, or orange flavouring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. The capsules of the present invention may comprise, in addition to the active, microcrystalline cellulose, poloxamer (e.g. poloxamer 407), sodium starch glycolate, colloidal silicon dioxide, and magnesium stearate.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid carrier, such as, for example, lactose, saccharose, sorbitol, mannitol, potato starch, corn starch, amylopectin, cellulose derivatives, or gelatin.

Tablets are solid pharmaceutical dosage forms containing therapeutic drug substances with or without suitable additives. Tablets are typically made by moulding, by compression or by generally accepted tablet forming methods. Compressed tablets are usually prepared by large-scale production methods while moulded tablets often involve small-scale operations.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

In general, there are three general methods of tablet preparation: (1) the wet-granulation method; (2) the dry-granulation method; and (3) direct compression. These methods are well known to those skilled in the art (see Remington's Pharmaceutical Sciences. 16th and 18th Eds., Mack Publishing Co., Easton, Pa. (1980 and 1990), and the U.S. Pharmacopeia XXI. U.S. Pharmacopeial Convention, Inc., Rockville, Md. (1985)).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be coloured and are beneficial in covering up drug substances possessing objectionable tastes or odours and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

In another aspect, the present invention provides a kit or article of manufacture comprising a pharmaceutical composition comprising a compound of formula (I), as described herein.

There is also provided a kit for use in a therapeutic or prophylactic application mentioned herein, the kit comprising: a container holding a pharmaceutical composition comprising a compound of formula (I); and a label or package insert with instructions for use.

Also provided is a kit comprising in separate compartments,

a first unit dosage form of the invention; and

a second unit dosage form of the invention,

wherein the kit is adapted to allow access to each compartment at a different time.

A “kit”, “kit of parts” or “article of manufacture” refers to a combination of components which may include a container for containing the pharmaceutical compositions and may also include divided containers such as a divided bottle or a divided foil packet, as well as a label or package insert on or associated with the container. The container can be in any conventional shape or form as known in the art that is made of a pharmaceutically acceptable material, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box.

The first and second unit dosage form of the kit may be any unit dosage form described herein. In some embodiments, the first and second unit dosage forms comprise the same amount of the compound of formula (I) or a pharmaceutically acceptable salt or prodrug thereof.

The kit may further comprise indicia distinguishing the first and second unit dosage forms from each other.

In some embodiments, the kit comprises a plurality of first unit dosage forms and a plurality of second unit dosage forms, each unit dosage forms within either plurality contained in a separate container adapted for to allow access at different times. For example the kit may comprise pluralities of the first and second unit dosage forms sufficient for 1, 2, 3, 4, 5, 6, 7, 8, 9, 10, 11, 12 or more weeks according to the determined dosage regimen.

The kit may further comprise instructions for coordinating the administration of the first and second dosage units as part of a treatment or prevention regimen comprising separate administration of a first unit dosage form and a second unit dosage form within a 24 hour period.

In some embodiments, the instructions coordinate the subject to take the first and second unit dosage forms with a high fat consumable. The high fat consumable may be any high fat consumable described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from a disorder described herein.

An example of such a kit in relation to solid dosage forms is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, veterinarian, pharmacist or other health care. When the kit contains separate compositions, a dose of one or more compositions of the kit can consist of one tablet, capsule, bottle, vial, or ampoule while a dose of another one or more compositions of the kit can consist of several tablets, capsules, bottles, vials or ampoules.

Uses

The present invention also relates to a method of treating or preventing a symptom associated with an endocrine disturbance in a subject in need thereof, the method comprising administering to the subject a pharmaceutical composition comprising a compound of formula (I) or a pharmaceutically acceptable salt or prodrug thereof and one or more pharmaceutically acceptable excipients.

The present invention also relates to the use of a pharmaceutical composition of the present invention in the manufacture of a medicament for treating or preventing a symptom associated with an endocrine disturbance.

The present invention also relates to the use of a pharmaceutical composition of the present invention for the treatment or prevention of a symptom associated with an endocrine disturbance in a subject.

The present invention relates to a pharmaceutical composition of the present invention for use in the treatment or prevention of a symptom associated with an endocrine disturbance in a subject.

The terms “treating”, “treatment” and “therapy” are used herein to refer to curative therapy. Therefore, in the context of the present disclosure, the term “treating” encompasses curing and ameliorating the severity of the symptom.

“Preventing” or “prevention” means preventing the occurrence of the symptom or tempering the severity of the symptom if it develops subsequent to the administration of the pharmaceutical compositions of the present invention. This prevents the onset of clinically evident symptoms altogether or the onset of a preclinically evident stage of symptoms in individuals at risk.

The subject may be suffering from, or at risk of suffering from, an endocrine disturbance. Endocrine disturbances include, without limitation, adrenal disorders, including adrenal insufficiencies such as Addison's disease, congenital adrenal hyperplasia and mineralocorticoid deficiency, Conn's syndrome, Cushing's syndrome, adrenogenital syndrome (including pheochromocytoma, adrenocortical carcinoma and glucocorticoid remediable aldosteronism), glucose homeostasis disorders (such as diabetes mellitus, hypoglycaemia, including idiopathic hypoglycemia and insulinoma), metabolic bone disease (including osteoporosis, osteitis deformans (Paget's disease of bone), rickets and osteomalacia), pituitary gland disorders (including diabetes insipidus, hypopituitarism, or panhypopituitarism, pituitary tumors, including pituitary adenomas, prolactinoma (or hyperprolactinemia), acromegaly, gigantism and Cushing's disease), parathyroid gland disorders (including primary hyperparathyroidism, secondary hyperparathyroidism, tertiary hyperparathyroidism and hypoparathyroidism, including pseudohypoparathyroidism), sex hormone disorders (including disorders of sex development or intersex disorders, hermaphroditism, gonadal dysgenesis and androgen insensitivity syndromes), hypogonadism (including gonadotropin deficiency, Kallmann syndrome, Klinefelter syndrome, ovarian failure, testicular failure and Turner syndrome), gender identity disorder, delayed or precocious puberty, menstrual function or fertility disorders (including amenorrhea and polycystic ovary syndrome), thyroid disorders (including goiter, hyperthyroidism, Graves' disease, thyroiditis and thyroid cancer), tumours of the endocrine glands (such as multiple endocrine neoplasia types 1, 2a and 2b), and autoimmune polyendocrine syndromes. Certain underlying disorders, such as HIV or certain cancers, can cause endocrine disturbances that lead to symptoms, including hot flashes, in the subject suffering from the disorder.

The symptom may be a hot flash. A “hot flash” or “hot flush” is a sudden temporary increase in body temperature: the “flash” sensation in a “hot flash” occurs as the body temperature peaks almost instantaneously and begins a much slower return to normal. Hot flashes can become so strong that they can raise the body temperature multiple degrees in a very short period of time, and cause the sufferer to feel weak and break out in heavy sweating. Successful treatment or prevention by a composition, method or use of the invention may result in fewer hot flashes over a given period or a reduction in the intensity of hot flashes.

Subjects may include but are not limited to primates, especially humans, domesticated companion animals such as dogs, cats, horses, and livestock such as cattle, pigs, sheep, with dosages as described herein.

The subject can be either a female or a male, although the ultimate cause of hot flashes can be markedly different for both groups. For example, in healthy female patients the hot flash is typically a primary symptom resulting from menopausal hormonal variation. However, the hot flash can also be drug-induced by anti-estrogen compounds (e.g., tamoxifen, leuprolide acetate, etc.) or surgically-induced by removal of estrogen-producing tissues (e.g. total abdominal hysterectomy, bilateral salpingo-oophorectomy, etc.). In male patients, the hot flashes typically occur as a side-effect of androgen-dependent therapy for metastatic prostate cancer. They can be either surgically-induced (e.g. bilateral orchiectomy) or drug-induced (e.g. treatment with a gonadotrophin-releasing-hormone agonist, leuprolide acetate, etc.).

The subject may be a menopausal or a premenopausal (also known as perimenopausal) female. Menopause is the point in a woman's life when she has not had a menstrual period for 1 year. For most women, menopause happens around age 50, but every woman's body has its own timeline. Some women stop having periods in their mid-40s. Others continue well into their 50s. Pre- or peri-menopause is the process of change that leads up to menopause. It can start as early as the late 30s or as late as the early 50s. How long premenopause lasts varies, but it usually lasts from 2 to 8 years. Symptoms associated with premenopause include changes in menstrual cycle, hot flashes, night sweats, vaginal dryness, sleep problems, mood changes (mood swings, sadness, or irritability), pain during sex, more urinary infections, urinary incontinence, less interest in sex, increase in body fat around the waist and problems with concentration and memory.

The subject may be diagnosed as being in need of treatment by suffering from at least one symptom associated with an endocrine disturbance, such as menopause. In particular, a subject is in need of treatment if they have suffered from at least one hot flash, in particular at least one hot flash within the six months prior to treatment. More particularly, the patient has suffered from at least one hot flash within three months, or within two months, or within one month prior to treatment.

The subject may be undergoing treatment with a therapeutic agent, and in particular with a chemotherapeutic agent. Women and men who have cancers, in particular those stimulated by sex hormones estrogen and androgen such as breast, uterine and testicular cancers, can get hot flashes as the chemotherapy lowers these body levels. In particular embodiments, the compound is administered to a subject at this will be, or is currently, receiving a chemotherapeutic treatment. The subject may be receiving a chemotherapeutic treatment within 10 days or within 9 days, or within 8 days or within 7 days, or within 6 days, or within 5 days or within 4 days, or within 3 days, or within 2 days or within one day or less of receiving the composition.

The composition may be administered after a chemotherapeutic treatment. The composition may be administered at least one hour after to, or at least two hours after, or at least three hours after, or at least four hours after, or at least five hours after, or at least six hours after, or at least seven hours after, or at least eight hours after, or at least twelve hours after, or at least one day after, the chemotherapeutic treatment.

The composition may be administered prior to, or concomitant with, a chemotherapeutic treatment. The composition may be administered at least one hour prior to, or at least two hours prior to, or at least three hours prior to, or at least four hours prior to, or at least five hours prior to, or at least six hours prior to, or at least seven hours prior to, or at least eight hours prior to, or at least twelve hours prior to, or at least one day prior to, the chemotherapeutic treatment.

The pharmaceutical composition may be administered once a day. The pharmaceutical composition may preferably be administered twice a day. The pharmaceutical composition may be administered with a high-fat consumable. The consumable may be a high-fat food or meal, or an edible fat or oil, such as fish oil. The compound of formula (I) may be administered to the subject in an amount of between about 50 mg and about 400 mg per dose. The amount may be between about 60 mg and about 350 mg, between about 70 mg and about 300 mg, between about 80 mg and about 250 mg, or between about 90 mg and about 200 mg. The amount may be about 100 mg per dose. The amount may be about 200 mg per dose. A 200 mg dose of a compound of formula (I) can be administered at one time (i.e. for once-daily, or QD, administration), or can be divided into two 100 mg doses for twice-daily (i.e. BID) administration. As used herein, the term “dose” or “dosage” refers to the amount of active i.e. compound of formula (I) that a subject takes or is administered at one time.

Administration and Pharmacokinetics

The present invention also provides for the administration of a compound of formula (I) to a subject, such as a subject experiencing a symptom associated with an endocrine disturbance, so as to obtain a desired pharmacokinetic profile, for example, a desired concentration of a compound of formula (I) in the plasma over a period of time. Such preferred pharmacokinetic profiles and/or endpoints may be achieved through the administration of specific doses, for example, 200 mg once a day or 100 mg twice a day, or may be achieved through the administration of doses individually-tailored for the specific recipient, taking into account factors such as weight, percent body fat, metabolism, ingestion of other therapeutic agents, etc.

Accordingly, in another aspect, the present invention relates to a pharmaceutical composition comprising an effective amount of a compound of formula (I) or a pharmaceutically acceptable salt or prodrug thereof.

and one or more pharmaceutically acceptable excipients,

wherein the pharmaceutical composition comprises a compound of formula (I) in an amount sufficient to provide a C_{trough} of about 150 to about 350 ng/mL when the composition is orally administered to a subject. The pharmaceutical composition may comprise a compound of formula (I) in an amount sufficient to provide a C_{trough} of about 150 to about 340, about 150 to about 330, about 150 to about 320, about 150 to about 310, or about 150 to 300, ng/mL when the composition is orally administered to a subject.

The pharmaceutical composition may comprise a compound of formula (I) or a pharmaceutically acceptable salt or prodrug thereof in an amount sufficient to provide a mean C_{trough} of about 230 ng/mL when the composition is orally administered to a subject. The pharmaceutical composition may provide a mean C_{trough} of about 225, about 226, about 227, about 228, about 229, about 230, about 231, about 232, about 233, about 234 or about 235, ng/mL when the composition is orally administered to a subject.

The pharmaceutical composition may comprise a compound of formula (I) or a pharmaceutically acceptable salt or prodrug thereof in an amount sufficient to provide a C_{trough} of greater than 130 ng/mL when the composition is orally administered to a subject. The pharmaceutical composition may comprise a compound of formula (I) in an amount sufficient to provide a C_{trough} of greater than about 135, about 140, about 145, about 150, or about 155, ng/mL when the composition is orally administered to a subject.

As used herein, the term “C_{trough}” refers to the minimum blood plasma concentration of the compound of formula (I) observed at steady state in a subject to which the compound of formula (I) has been administered. As used herein, the term “steady state” refers to the time period between 5 and 7 t_{1/2} from first administration of the compound of formula (I).

The pharmaceutical composition may comprise a compound of formula (I) or a pharmaceutically acceptable salt or prodrug thereof in an amount sufficient to provide a Cₘₐₓ of about 670 to about 1300 ng/mL when the composition is orally administered to a subject. The pharmaceutical composition may comprise a compound of formula (I) in an amount sufficient to provide a Cₘₐₓ of about 650 to about 1500, about 660 to about 1400, or about 670 to about 1300 ng/mL when the composition is orally administered to a subject.

The pharmaceutical composition may comprise a compound of formula (I) or a pharmaceutically acceptable salt or prodrug thereof in an amount sufficient to provide a mean Cₘₐₓ of about 1000 ng/mL when the composition is orally administered to a subject. The pharmaceutical composition may comprise a compound of formula (I) in an amount sufficient to provide a mean Cₘₐₓ of about 960, about 970, about 980, about 990, about 1000, about 1010, about 1020, about 1030, about 1040, or about 1050, ng/mL when the composition is orally administered to a subject.

As used herein, the term “Cₘₐₓ” refers to the maximum blood plasma concentration of the compound of formula (I) observed in a subject to which the compound of formula (I) has been administered.

The pharmaceutical composition may comprise a compound of formula (I) or a pharmaceutically acceptable salt or prodrug thereof in an amount sufficient to provide an AUC_{0-inf} of about 5500 to about 9500 h*ng/mL when the composition is orally administered to a subject. The pharmaceutical composition may comprise a compound of formula (I) in an amount sufficient to provide an AUC_{0-inf} of about 5600 to about 9400, about 5700 to about 9300, about 5800 to about 9200, or about 5900 to about 9100, ng/mL when the composition is orally administered to a subject.

The pharmaceutical composition may comprise a compound of formula (I) or a pharmaceutically acceptable salt or prodrug thereof in an amount sufficient to provide a mean AUC_{0-inf} of about 7200 ng/mL when the composition is orally administered to a subject. The pharmaceutical composition may comprise a compound of formula (I) in an amount sufficient to provide a mean Cₘₐₓ of about 7000, about 7100, about 7200, about 7300, or about 7400, h*ng/mL when the composition is orally administered to a subject.

As used herein, the term “AUC_{0-inf}” refers to the area under the plasma concentration-time curve from time 0 to infinite time. It is calculated as the sum of AUCₗₐₛₜ (i.e. the last quantifiable AUC of the compound of formula (I)) and Cₗₐₛₜ/kel (where Cₗₐₛₜ is the last observed quantifiable concentration in plasma, and kel is the apparent terminal elimination rate constant).

The subject may be a fed subject. The consumable that the subject has been fed may be a high-fat consumable. The high-fat consumable may be a high-fat food or meal, or an edible fat or oil, such as fish oil.

The present inventors have found that trough concentrations with BID (i.e. twice-daily) dosing are approximately double those of QD (i.e. once daily) dosing. As shown in the Examples, the Cₘᵢₙ concentrations upon 200 mg QD dosing were 52-94 ng/mL (mean 73 ng/mL), whereas those for 100 mg BID were 117-195 ng/mL (mean 146 ng/mL). If exposure in patients is lower than in healthy subjects, the trough levels at 200 mg QD may approach—or be lower than—those required for efficacy. Given the likelihood of high inter-individual variability in a patient population, and the fact that patients (particularly oncology patients) may not always take the medication with food (thereby decreasing their exposure), there is significant potential for patients receiving 200 mg QD to have low trough concentrations, which might not be consistent with duration of action throughout the dosing interval. The present inventors therefore believe that twice-daily dosing at 100 mg provides greater certainty of efficacy in a greater number of subjects given the likely high variability in exposure (and particularly trough concentrations) driven by disease and other factors, such as the potential for dosing without food.

The present invention also relates to a method of increasing the bioavailability of a compound of formula (I), comprising administering to a subject in need thereof a pharmaceutical composition of the present invention, wherein the pharmaceutical composition is administered with food. The present inventors believe that the bioavailability of the compound of formula (I) is improved in the presence of food due to the hydrophobic nature of the compound of formula (I). The consumable may be a high-fat food or meal, or an edible fat or oil, such as fish oil. The pharmaceutical composition of the present invention may be administered once a day with food. The pharmaceutical composition may be administered twice a day (for example, in the morning and in the evening) with food.

Levels of the compound of formula (I) in the plasma may be assessed by any art-accepted method. Determination of the concentration of the compound of formula (I) in the plasma may be accomplished as follows. Following administration of the compound, samples of blood are taken at specific time points. The samples are centrifuged and the plasma supernatant is removed and stored at −80° C. The compound and the Internal Standard (IS) can be obtained from human plasma using protein precipitation extraction. The analytes are separated by HPLC, and the eluates monitored by a MS/MS detector in positive ion mode. The extract is then assayed against a calibration curve.

As shown in the Examples, a high-fat meal led to a clinically meaningful compound of formula (Ia) exposure increase. Compared to the fasted state, a high-fat meal increased mean Cₘₐₓ and mean AUC_{0-inf} of a single oral dose of the compound of formula (Ia) (200 mg) by about 60% and 45%, respectively.

Accordingly, the pharmaceutical composition of the present invention, as described herein, may result in an increase in mean Cₘₐₓ of the compound of formula (I) by about 50%, by about 55%, by about 60%, by about 65%, or by about 70%, when orally administered with a high-fat consumable, compared to administration in a fasted state.

The pharmaceutical composition of the present invention, as described herein, may result in an increase in mean Cₘₐₓ of the compound of formula (I) by at least about 50%, about 55%, about 60%, about 65%, or about 70%, when orally administered with a high-fat consumable, compared to administration in a fasted state.

The pharmaceutical composition of the present invention, as described herein, may result in an increase in mean AUC_{0-inf} of the compound of formula (I) by about 40%, about 45%, about 50%, about 55%, or about 60%, when orally administered with a high-fat consumable, compared to administration in a fasted state.

The pharmaceutical composition of the present invention, as described herein, may result in an increase in mean AUC_{0-inf} of the compound of formula (I) by at least about 50%, about 55%, about 60%, about 65%, or about 70%, when orally administered with a high-fat consumable, compared to administration in a fasted state.

In addition, surprisingly, when the pharmaceutical composition is administered twice a day with food, particularly a high-fat food or meal, the C_{avg} doubles in some subjects (compared to once-a-day dosing of the equivalent amount) but the Cₘₐₓ increases less than two-fold (C_{avg} is the average steady state concentration over a steady state dosing interval, calculated as AUC₀₋ₜₐᵤ/tau). This is a significant advantage as it means that the compound of formula (I) accumulates and therefore maintains therapeutic plasma levels, while not significantly affecting the maximum plasma concentration, thereby avoiding potential problems such as undesired side-effects, overdosing and adverse toxicological effects that may occur with higher drug concentrations.

As used herein, terms such as “administration with food”, “administered with food”, “administered to a fed subject”, and the like, refer to the administration of the pharmaceutical composition of the present invention at any time relative to the consumption of the food, provided that the consumed food results in an increased exposure to the compound of formula (Ia). The pharmaceutical composition may be administered at the same time, shortly before, or after eating of the food. Preferably, the subject administers the pharmaceutical composition within 30 minutes of the time the subject began to ingest the food. Preferably, the subject will administer the pharmaceutical composition within 10 minutes of completing the food.

Embodiments of the invention will now be discussed in more detail with reference to the examples which are provided for exemplification only and which should not be considered limiting on the scope of the invention in any way.

### EXAMPLES

Pharmacokinetic (PK) Study

A PK study was conducted to assess the effect of food and determine whether once- or twice-daily dosing of the compound of formula (Ia) would be beneficial to a subject.

Part 1 involved analysing the PK parameters obtained following single oral administration of 200 mg tablets of a compound of formula (Ia) in free base form to 12 healthy female subjects following either fed (high-fat meal) or fasted conditions.

Part 2 involved analysing the PK parameters obtained following either single (QD) or twice daily (BD) dosing of a compound of formula (Ia) under fed conditions.

Q-122 was supplied as 100 and 200 mg capsules (described above) in HDPE bottles.

The relevant PK parameters assessed were:

For Part 1
Cmax maximum plasma concentration (observed)
Tmax time to reach the maximum plasma concentration (observed)
Clast last observed quantifiable concentration in plasma
Tlast time of last observed quantifiable concentration in plasma
AUC0-last area under the plasma concentration-time curve from time 0 to the time of the last quantifiable concentration
AUCinf area under the plasma concentration-time curve from time 0 to infinite time, calculated as the sum of AUCₗₐₛₜ and Cₗₐₛₜ/kel
% AUCexp percent of AUC extrapolated between AUC₀₋ₗₐₛₜ and AUC_{inf}
kel apparent terminal elimination rate constant
t_{1/2} elimination half-life associated with the terminal slope (kel) of the semilogarithmic drug concentration-time curve, calculated as 0.693/kel
CL/F apparent total body clearance of a drug from the plasma calculated by: CL/F=Dose/AUC_{inf}
Vz/F apparent volume of distribution at steady state calculated by: V/F=kel*(Dose/AUC_{inf})

For Part 2
Cmax maximum plasma concentration (observed)
Ctrough plasma concentration observed just before treatment administration during repeated dosing (depicted in plots
Tmax time to reach the maximum plasma concentration (observed)
Cmin Minimum concentration between dose time and dose time+tau time of last observed quantifiable concentration in plasma
AUC0-last area under the plasma concentration-time curve from time 0 to the time of the last quantifiable concentration
AUC0-tau AUC from time zero to the last quantifiable concentration. Where tau is the dosing interval of either 12 or 24 hours (Day 10 only)
AUCinf AUC from time 0 to infinite time, calculated as the sum of AUCₗₐₛₜ and Cₗₐₛₜ/Kel (Day 10 only)
% AUCexp percent of AUC extrapolated between AUC₀₋ₗₐₛₜ and AUC_{inf}
kel apparent terminal elimination rate constant
t_{1/2} elimination half-life associated with the terminal slope (kel) of the semilogarithmic drug concentration-time curve, calculated as 0.693/Kel (Day 10 only)
CL/F apparent total body clearance of a drug from the plasma calculated by: CL/F=Dose/AUC_{inf}
Vz/F apparent volume of distribution at y state calculated by: V/F=Kel*(Dose/AUC_{inf})
Cavg average steady state concentration over a steady state dosing interval: AUC0-tau/tau
% Fluctuation 100*(Cmax−Cmin)/Cavg
Accumulation index 1/[1−e\hat{ }^{Kel*tau}] where tau is the dosing interval

Method

Part 1

An initial dose was administered on Day 1 followed by collection of samples for PK analysis for 96 hours. PK blood samples were taken pre-dose and 0.25, 0.5, 1.0, 1.5, 2, 3, 4, 8, 10, 12, 16, 24, 36, 48, 60, 72 and 96 hours post-dose, and urine samples were taken at 0-4, 4-8, 8-12, 12-24 and 24-48 hours. Ten days after the initial dose of study drug (Day 11), a second dose of drug was administered followed by collection of PK samples. Subjects completed the End-of-Study Visit (EOS) on Day 18, 7 days after the last dose of study drug.

Subjects selected for participation checked into the clinical unit and were randomized (on the morning of Day 1) to food state for study treatment; one half received study drug in the fed state first followed by fasted state for the second dose while the other half received study drug in the fasted state first followed by fed state. Prior to dosing on Day 1, all subjects fasted for a minimum of 10 hours overnight. On the morning of Day 1, subjects dosed in the fed state received the study drug within 10 min of completing a high fat breakfast. Subjects dosed in the fasted state were dosed early in the morning and did not receive food until 4 hours after dosing. Plasma and urine samples were collected as set out above for the PK analysis. Subjects remained in the clinic for study procedures and blood draws before checking out after completion of study procedures on the morning of Day 4 (i.e., 72 hours post-dose) returning for the final PK blood draw on Day 5 at 96 hr post-dose. After the first single dose of study drug, subjects completed a 10-day washout period, returning to the clinical unit for check-in on Day 10. Subjects fasted overnight, receiving the second dose of study drug in the fed or fasted state on the morning of Day 11. Subjects were discharged from the clinic after the collection of the final 72-hr PK sample returning the next day for the 96 hr blood draw.

Part 2

Subjects were randomized 1:1 to receive a daily dose of 200 mg administered once daily in the morning (200 mg capsule) or twice daily (100 mg capsules BID) for a total of 10 days. PK blood samples were taken on Day 1 pre-dose and at 0.25, 0.5, 1.0, 1.5, 2, 3, 4, 8, 10, 12 and 16 hours post-dose. On Days 2 to 10, PK blood samples were taken once pre-dose, and on Days 11 to 14, at 24, 36, 48, 60, 72 and 96 hours post-dose.

Subjects selected for participation checked into the clinical unit and were randomized to the dosing regimen receiving their first dose of study drug on the morning of Day 1 followed by PK and safety assessments through the morning of Day 2 (24 hours post-dose). Subjects on a BID regimen received their second dose of study drug approximately 12 hours after the first dose. Subjects remained confined in the clinic for dosing BID or QD through the last dose of study drug on the morning of Day 10. PK samples were collected through 96 hrs post-dose. Subjects were discharged from the clinic following collection of the 72 hr PK sample, returning to the clinic on the morning of Day 14 for the 96 hr sample collection.

For both Parts 1 and 2, samples were processed and analysed as follows. The method extracted Q-122 and the Internal Standard (IS, Q-122 ¹³C₄) from human plasma using protein precipitation extraction.

Control human plasma (K₂EDTA anticoagulant) was received from Bioreclamation. It was stored in a monitored freezer at nominal −20° C. prior to use for the preparation of plasma calibration curve and quality control samples.

Q-122 stock solutions were prepared in duplicate in N,N-dimethylacetamide (DMA) at 1.00 mg/mL. The solutions were verified against each other and deemed acceptable for use (within 5% of each other). The individual stock solutions were then mixed to create a combined stock solution. All stock solutions were stored in amber glass vials at room temperature and used within a valid stability timeframe.

The combined stock solution was diluted with 50% methanol/0.1% TFA (DSA) to prepare calibration curve working solutions within the range 800 to 100,000 ng/mL. Calibration curve working solutions were stored in polypropylene tubes at room temperature and used within a valid stability timeframe.

Plasma calibration curve samples were prepared in bulk by spiking calibration curve working solutions into control human plasma containing K₂EDTA. Calibration curve samples were stored in a monitored freezer at nominal −80° C. prior to use. Plasma calibration curve standard samples were prepared at the following concentrations: 10.0, 20.0, 50.0, 200, 800, 1250, 2250, 2500 ng/mL. All plasma calibration curve samples were used within a valid stability timeframe.

The combined stock solution was diluted with 50% methanol/0.1% TFA (DSA) to prepare quality control working solutions within the range 1000 to 100,000 ng/mL. Quality control working solutions were stored in polypropylene tubes at room temperature and used within a valid stability timeframe.

Plasma quality control samples were prepared in by spiking quality control working solutions into control human plasma containing K₂EDTA. Quality control samples were stored in a monitored freezer at nominal −80° C. prior to use. Plasma quality control samples used in this project were prepared at the following concentrations: 30.0 (PQCL), 500 (PQCM), 2000 (PQCH) ng/mL. All plasma quality control samples were used within a valid stability timeframe.

The analytes were separated by HPLC on a Phenomenex Synergi Polar-RP column, and the eluates monitored by an API4000 MS/MS detector in positive MRM mode. The extract was then assayed against a calibration curve. The data were acquired and processed by the data acquisition system Analyst® (Sciex) linked directly to the API4000 MS/MS detector and then processed in Watson LIMS™ (Thermo Scientific), where applicable. The method range is from 10.0 to 2500 ng/mL using 50 μL of matrix and has a run time of approximately 3.2 minutes per sample.

All primary samples were analysed as received, with all samples of the same type from the same period from an individual subject analysed within the same analytical run (except for repeat samples and ISR). System suitability testing was performed and deemed acceptable prior to sample analytical runs being acquired.

The plasma samples were analysed together with the following samples:
Reagent blank sample
Matrix blank sample
Zero sample (Matrix plus internal standard)
Calibration curve samples in duplicate, one set in increasing concentration order at the beginning of the run and one set in decreasing concentration order at the end of the run
At least duplicate PQCL, PQCM, PQCH samples

Quality control samples were randomly dispersed throughout the analytical run, with test samples being bound by calibration curve samples.

Data were collected and integrated using Analyst® (Sciex), then regression and reporting performed using Watson LIMS™ (Thermo Scientific), where appropriate. Peak area ratios (analyte: internal standard) using linear regression with 1/x² weighting were used to generate calibration curves for the analyte.

Plasma PK parameters for each dosing regimen and dose level were calculated from the concentrations of Q-122 and its major metabolites measured in pre-dose and post-dose plasma samples. For each dose level, descriptive statistics (sample size, arithmetic means, geometric means, standard deviation, % coefficient of variation, minimum, median, and maximum) are presented.

The pharmacokinetic results were processed according to standard non-compartmental analytical procedures. The software used was Phoenix™ WinNonlin® v 6.4 (Pharsight Corporation, USA) and Microsoft® Excel® 2010 (Microsoft Corporation). The following PK parameters were estimated from plasma data.

Results

Part 1

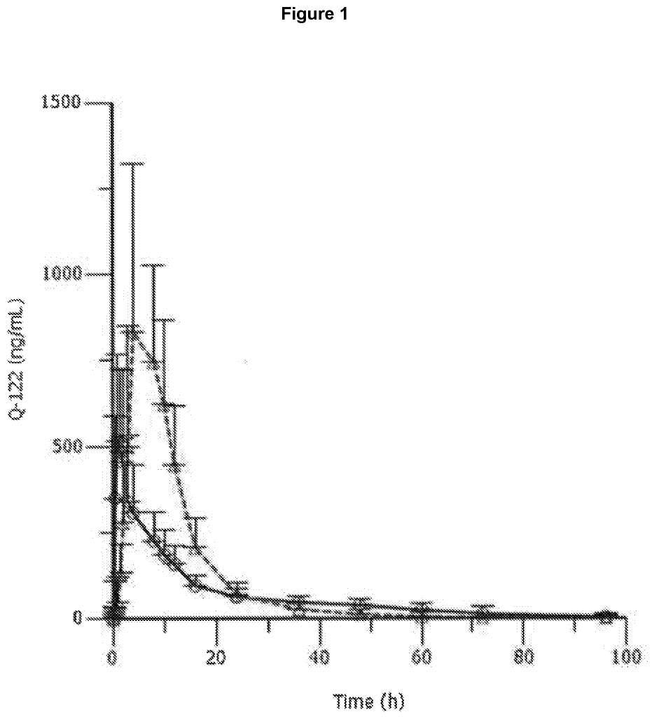

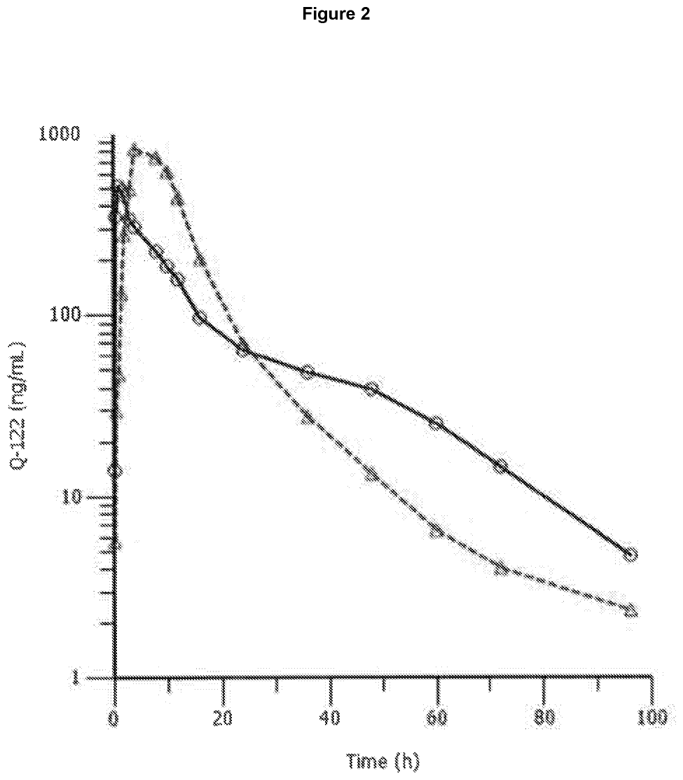

The results of this study are given in Tables 1 and 2 below, as well as FIGS. 1 and 2.

TABLE 1
Fasted for Part 1 (200 mg)

| Food | | Tₘₐₓ | Cₘₐₓ | Tₗₐₛₜ | Cₗₐₛₜ | AUC₀₋ₗₐₛₜ | AUC_{inf} | % AUCexp | Vz/F | Cl/F | kel | t_{1/2} |
|---|---|---|---|---|---|---|---|---|---|---|---|---|
| status | Subject | (h) | (ng/ml) | (h) | (ng/mL) | (h*ng/mL) | (h*ng/mL) | (%) | (L) | (L/h) | (1/h) | (h) |
| Fasted | 101001 | 0.50 | 559.00 | 72.00 | 39.70 | 5150.3 | ND | 30.9 | ND | ND | ND | ND |
| | 101002 | 1.00 | 1050.00 | 72.00 | 16.30 | 9154.4 | 9726.1 | 5.9 | 721.24 | 20.56 | 0.029 | 24.31 |
| | 101003 | 0.50 | 615.00 | 60.00 | 11.40 | 6812.7 | 7031.1 | 3.1 | 544.90 | 28.45 | 0.052 | 13.28 |
| | 101004 | 1.50 | 422.00 | 24.00 | 22.00 | 3704.2 | Terminal phase inaccurate | | | | | |
| | 101005 | 2.00 | 611.00 | 48.00 | 15.50 | 5881.1 | 6144.8 | 4.3 | 553.84 | 32.55 | 0.059 | 11.79 |
| | 101006 | 1.00 | 801.00 | 96.00 | 26.40 | 9258.3 | 10317.1 | 10.3 | 777.51 | 19.39 | 0.025 | 27.80 |
| | 101007 | 1.50 | 676.00 | 96.00 | 16.30 | 7702.0 | 8470.5 | 9.1 | 1113.30 | 23.61 | 0.021 | 32.68 |
| | 101008 | 2.00 | 1010.00 | 60.00 | 11.70 | 8375.7 | 8608.7 | 2.7 | 462.64 | 23.23 | 0.050 | 13.80 |
| | 101009 | 8.00 | 103.00 | 96.00 | 14.20 | 4924.9 | 5415.4 | 9.1 | 1275.63 | 36.93 | 0.029 | 23.94 |
| | 101010 | 0.50 | 516.00 | 60.00 | 22.40 | 5410.8 | 6250.2 | 13.4 | 1199.14 | 32.00 | 0.027 | 25.98 |
| | 101011 | 1.50 | 668.00 | 60.00 | 30.90 | 6774.9 | 7756.0 | 12.6 | 818.75 | 25.79 | 0.031 | 22.01 |
| | 101012 | 1.00 | 403.00 | 60.00 | 17.70 | 3631.6 | 4092.4 | 11.3 | 1272.25 | 48.87 | 0.038 | 18.04 |
| | N | 12 | 12 | 12 | 12 | 12 | 10 | 11 | 10 | 10 | 10 | 10 |
| | Mean | 1.750 | 619.500 | 67.000 | 20.375 | 6398.39 | 7381.22 | 10.23 | 873.921 | 29.137 | 0.0361 | 21.364 |
| | SD | 2.039 | 259.969 | 21.380 | 8.450 | 1944.28 | 1959.69 | 7.79 | 315.983 | 8.918 | 0.0131 | 6.926 |
| | Min | 0.50 | 103.00 | 24.00 | 11.40 | 3631.6 | 4092.4 | 2.7 | 462.64 | 19.39 | 0.021 | 11.79 |
| | Median | 1.25 | 613.00 | 60.00 | 17.00 | 6327.98 | 7393.54 | 9.07 | 798.13 | 27.12 | 0.0302 | 22.97 |
| | Max | 8.00 | 1050.00 | 96.00 | 39.70 | 9258.3 | 10317.1 | 30.9 | 1275.63 | 48.87 | 0.059 | 32.68 |
| | CV % | 116.5 | 42.0 | 31.9 | 41.5 | 30.4 | 26.5 | 76.1 | 36.2 | 30.6 | 36.2 | 32.4 |

Note:
ND = not determined
Bolded number indicates a Tₗₐₛₜ of 24 h

TABLE 2
Fed for Part 1 (200 mg)

| Food | | Tₘₐₓ | Cₘₐₓ | Tₗₐₛₜ | Cₗₐₛₜ | AUC₀₋ₗₐₛₜ | AUCinf | % AUCexp | Vz/F | Cl/F | kel | t_{1/2} |
|---|---|---|---|---|---|---|---|---|---|---|---|---|
| status | Subject | (h) | (ng/mL) | (h) | (ng/mL) | (h*ng/mL) | (h*ng/mL) | (%) | (L) | (L/h) | (1/h) | (h) |
| Fed | 101001 | 8.00 | 914.00 | 48.00 | 15.40 | 11821.5 | 12030.1 | 1.7 | 225.16 | 16.62 | 0.074 | 9.39 |
| | 101002 | 4.00 | 1240.00 | 48.00 | 13.60 | 12551.6 | 12760.3 | 1.6 | 240.47 | 15.67 | 0.065 | 10.63 |
| | 101003 | 8.00 | 1230.00 | 48.00 | 10.90 | 14254.9 | 14389.6 | 0.9 | 171.77 | 13.90 | 0.081 | 8.57 |
| | 101004 | 8.00 | 779.00 | 36.00 | 12.20 | 6505.3 | 6606.2 | 1.5 | 250.37 | 30.27 | 0.121 | 5.73 |
| | 101005 | 4.00 | 1520.00 | 36.00 | 11.00 | 11087.2 | 11171.4 | 0.8 | 136.98 | 17.90 | 0.131 | 5.30 |
| | 101006 | 4.00 | 1180.00 | 96.00 | 14.70 | 12782.0 | 13417.1 | 4.7 | 638.99 | 14.91 | 0.023 | 29.71 |
| | 101007 | 0.50 | 267.00 | 96.00 | 11.20 | 4088.9 | 4422.3 | 7.5 | 1346.32 | 45.22 | 0.034 | 20.63 |
| | 101008 | 8.00 | 797.00 | 24.00 | 34.20 | 8498.9 | ND | 2.0 | Terminal phase inaccurate | | | |
| | 101009 | No data available | | | | | | | | | | |
| | 101010 | 10.00 | 929.00 | 48.00 | 15.10 | 12043.9 | 12197.9 | 1.3 | 167.23 | 16.40 | 0.098 | 7.07 |
| | 101011 | 10.00 | 603.00 | 60.00 | 16.70 | 7713.8 | 8201.3 | 5.9 | 711.86 | 24.39 | 0.034 | 20.23 |
| | 101012 | 4.00 | 1400.00 | 36.00 | 21.50 | 11327.4 | 11516.0 | 1.6 | 152.37 | 17.37 | 0.114 | 6.08 |
| | N | 11 | 11 | 11 | 11 | 11 | 10 | 11 | 10 | 10 | 10 | 10 |
| | Mean | 6.227 | 987.182 | 52.364 | 16.045 | 10243.66 | 1067.21 | 2.70 | 404.151 | 21.266 | 0.0775 | 12.336 |
| | SD | 3.061 | 369.983 | 23.560 | 6.775 | 3111.30 | 3206.68 | 2.28 | 389.429 | 9.790 | 0.0386 | 8.293 |
| | Min | 0.50 | 267.00 | 24.00 | 10.90 | 4088.9 | 4422.3 | 0.8 | 136.98 | 13.90 | 0.023 | 5.30 |
| | Median | 8.00 | 929.00 | 48.00 | 14.70 | 11327.40 | 11773.06 | 1.64 | 232.81 | 17.00 | 0.0774 | 8.98 |
| | Max | 10.00 | 1520.00 | 96.00 | 34.20 | 14254.9 | 14389.6 | 7.5 | 1346.32 | 45.22 | 0.131 | 29.71 |
| | CV % | 49.2 | 37.5 | 45.0 | 42.2 | 30.4 | 30.0 | 84.4 | 96.4 | 46.0 | 49.8 | 67.2 |

Plasma profiles of Q-122 following administration of a single dose of 200 mg to subjects under fed and fasting conditions have been determined according to the protocol discussed above. Under fasted conditions, rapid absorption was apparent following single dose (Tₘₐₓ of 0.5-8 hour) compared to under fed conditions (0.5-10 h). However, on omission of subject 101009 from the fasted cohort, the range for Tₘₐₓ was (0.5-2 h) for fasted conditions. The Cₘₐₓ and AUC₀₋ₗₐₛₜ were approximately doubled in 6 out of 11 subjects following food. Mean results indicated a 1.6-fold increase in Cₘₐₓ and AUC after food. Apparent volume of distribution and total body clearance ranged between similar values regardless of fed or fasted status (Table 1). Similar levels of variability were observed between the two cohorts for Cₘₐₓ and AUC parameters.

In conclusion, it appears that administration of 200 mg Q-122 with a fatty meal results in an increase in bioavailability.

Part 2

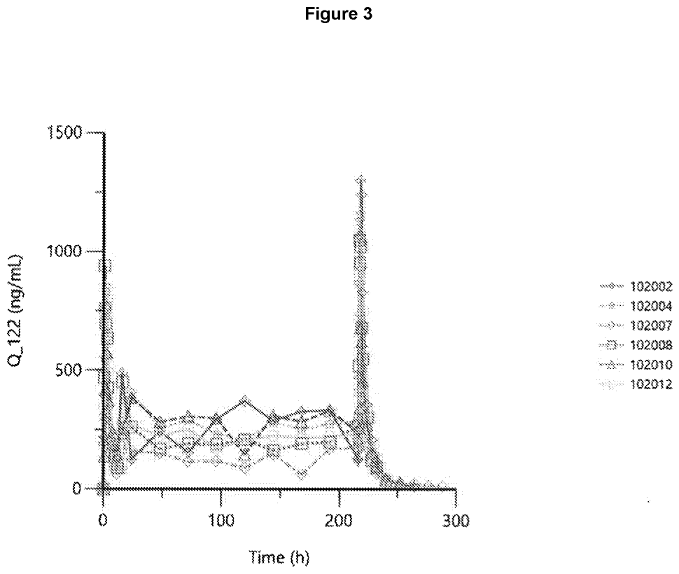

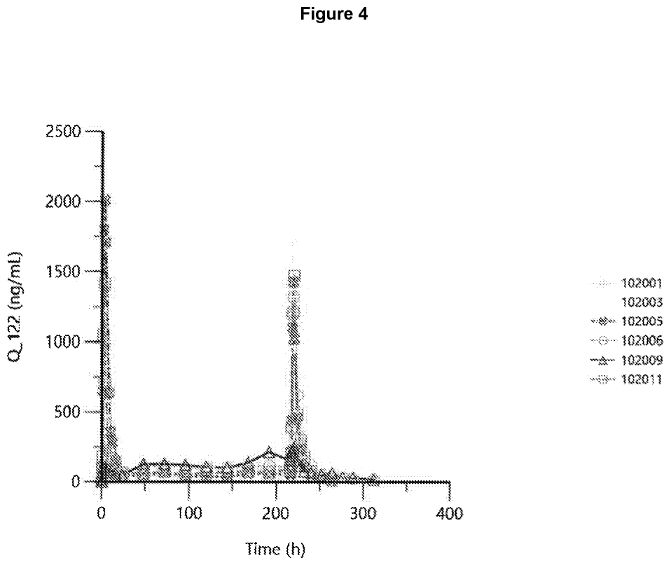

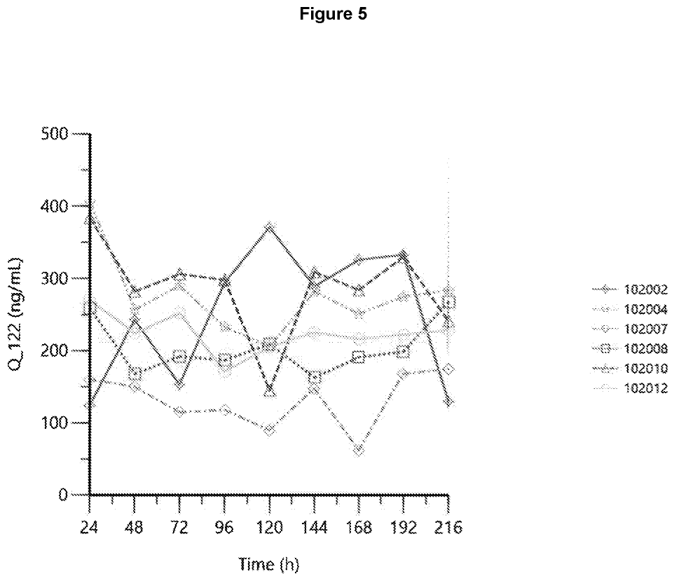

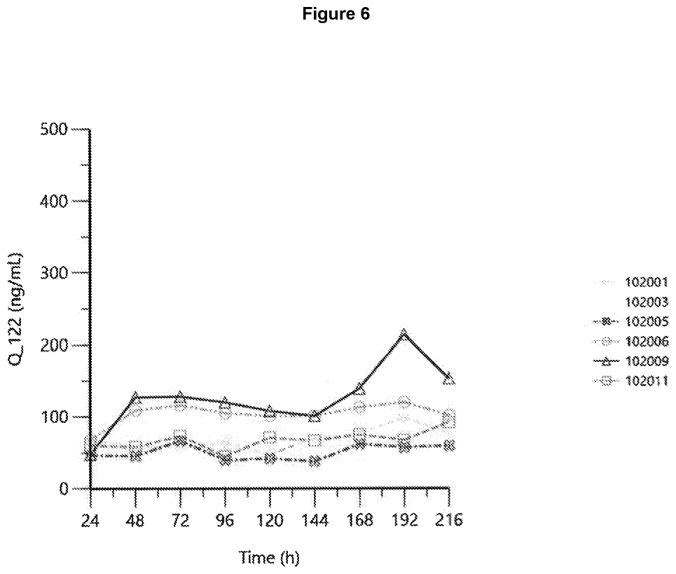

The results of this study are given in Tables 3 and 4 below, as well as FIGS. 3, 4, 5 and 6.

TABLE 3
100 mg administered BID (Part 2) Updated

| Dosing | Subject | Tmax | Cmax | AUC0-last | AUCinf | % AUCexp | Cavg | Fluct |
|---|---|---|---|---|---|---|---|---|
| Day | Number | (h) | (ng/mL) | (h*ng/mL) | (h*ng/mL) | (%) | (ng/mL) | % |
| 1 | 102002 | 4.00 | 405.00 | 6463 | ND | ND | 232.90 | 173.90 |
| | 102004 | 1.50 | 852.00 | 6090 | ND | ND | 315.05 | 270.44 |
| | 102007 | 3.00 | 563.00 | 5186 | ND | ND | 227.25 | 247.74 |
| | 102008 | 1.00 | 939.00 | 8167 | ND | ND | 354.49 | 264.89 |
| | 102010 | 2.00 | 773.00 | 6822 | ND | ND | 299.97 | 257.70 |
| | 102012 | 2.00 | 845.00 | 7028 | ND | ND | 364.87 | 231.59 |
| | N | 6 | 6 | 6 | 6 | 6 | 6 | 6 |
| | Mean | 2.250 | 729.580 | 6626.1 | ND | ND | 299.088 | 241.041 |
| | SD | 1.084 | 203.544 | 996.0 | ND | ND | 58.642 | 35.644 |
| | Min | 1.00 | 405.00 | 5186 | ND | ND | 227.25 | 173.90 |
| | Median | 2.00 | 809.00 | 6642.2 | ND | ND | 307.51 | 252.72 |
| | Max | 4.00 | 939.00 | 816.7 | ND | ND | 364.87 | 270.44 |
| | CV % | 48.2 | 27.9 | 15.0 | ND | ND | 19.6 | 14.8 |
| 10 | 102002 | 2.00 | 1300.00 | 8812 | 9015 | 2.24 | 583.47 | 201.90 |
| | 102004 | 2.00 | 1160.00 | 7715 | 7834 | 1.52 | 498.51 | 199.80 |
| | 102007 | 1.50 | 865.00 | 5510 | 6000 | 8.16 | 310.92 | 240.58 |
| | 102008 | 1.00 | 1050.00 | 6477 | 6779 | 4.45 | 462.09 | 200.83 |
| | 102010 | 2.00 | 677.00 | 5615 | 6059 | 7.33 | 343.13 | 152.13 |
| | 102012 | 2.00 | 1070.00 | 7597 | 7945 | 4.38 | 523.54 | 167.13 |
| | N | 6 | 6 | 6 | 6 | 6 | 6 | 6 |
| | Mean | 1.750 | 1020.333 | 6954.5 | 7271.9 | 4.679 | 453.609 | 193.726 |
| | SD | 0.418 | 220.487 | 1307.8 | 1194.9 | 2.653 | 106.217 | 30.941 |
| | Min | 1.00 | 677.00 | 5510 | 6000 | 1.52 | 310.92 | 152.13 |
| | Median | 2.00 | 1060.00 | 7037.2 | 7306.5 | 4.42 | 480.30 | 200.31 |
| | Max | 2.00 | 1300.00 | 8812 | 9015 | 8.16 | 583.47 | 240.58 |
| | CV % | 23.9 | 21.6 | 18.8 | 16.4 | 56.7 | 23.4 | 16.0 |

| Dosing | Subject | Accum | t½ | AUC0-tau | Vz/F | CLss/F | Cmin | Tlast |
|---|---|---|---|---|---|---|---|---|
| Day | Number | Index | (h) | (h*ng/mL) | (L) | (L/h) | (ng/mL) | (h) |
| 1 | 102002 | ND | 9.38 | 2795 | ND | ND | ND | 24.00 |
| | 102004 | ND | 6.21 | 3781 | ND | ND | ND | 24.00 |
| | 102007 | ND | 3.97 | 2727 | ND | ND | ND | 24.00 |
| | 102008 | ND | 3.19 | 4254 | ND | ND | ND | 24.00 |
| | 102010 | ND | 5.52 | 3600 | ND | ND | ND | 24.00 |
| | 102012 | ND | 4.66 | 4378 | ND | ND | ND | 24.00 |
| | N | 6 | 6 | 6 | 6 | 6 | 6 | 6 |
| | Mean | ND | 5.488 | 3589.1 | ND | ND | ND | 24.000 |
| | SD | ND | 2.188 | 703.7 | ND | ND | ND | 0.000 |
| | Min | ND | 3.19 | 2727 | ND | ND | ND | 24.00 |
| | Median | ND | 5.1 | 3690.08 | ND | ND | ND | 24.00 |
| | Max | ND | 9.38 | 4378 | ND | ND | ND | 24.00 |
| | CV % | ND | 39.9 | 19.6 | ND | ND | ND | 0.0 |
| 10 | 102002 | 2.06 | 12.52 | 7002 | 257.95 | 14.28 | 122.00 | 48.00 |
| | 102004 | 1.33 | 6.01 | 5982 | 144.95 | 16.72 | 164.00 | 36.00 |
| | 102007 | 4.17 | 30.30 | 3731 | 1171.51 | 26.80 | 117.00 | 72.00 |
| | 102008 | 1.36 | 6.26 | 5545 | 162.90 | 18.03 | 122.00 | 24.00 |
| | 102010 | 1.79 | 10.19 | 4118 | 356.99 | 24.29 | 155.00 | 36.00 |
| | 102012 | 1.28 | 5.46 | 6283 | 125.31 | 15.92 | 195.00 | 24.00 |
| | N | 6 | 6 | 6 | 6 | 6 | 6 | 6 |
| | Mean | 1.998 | 11.789 | 5443.3 | 369.935 | 19.340 | 145.833 | 40.000 |
| | SD | 1.105 | 9.483 | 1274.6 | 402.133 | 5.021 | 30.980 | 18.087 |
| | Min | 1.28 | 5.46 | 3731 | 125.31 | 14.28 | 117.00 | 2400 |
| | Median | 1.58 | 8.2 | 5763.63 | 210.42 | 17.38 | 138.50 | 36.00 |
| | Max | 4.17 | 30.30 | 7002 | 1171.51 | 26.80 | 195.00 | 72.00 |
| | CV % | 55.3 | 80.4 | 23.4 | 108.7 | 26.0 | 21.2 | 45.2 |

TABLE 4
200 mg administered QD (Part 2) Updated

| Dosing | Subject | Tmax | Cmax | AUC0-last | AUCinf | % AUCexp | Cavg | Fluct |
|---|---|---|---|---|---|---|---|---|
| Day | Number | (h) | (ng/mL) | (h*ng/mL) | (h*ng/mL) | (%) | (ng/mL) | % |
| 1 | 102001 | 4.00 | 1700.00 | 8831 | ND | ND | 367.97 | 461.99 |
| | 102003 | 2.00 | 1880.00 | 12137 | ND | ND | 505.70 | 371.76 |
| | 102005 | 3.00 | 2010.00 | 12571 | ND | ND | 523.80 | 383.73 |
| | 102006 | 3.00 | 980.00 | 6439 | ND | ND | 268.28 | 365.28 |
| | 102009 | 4.00 | 101.00 | 1632 | ND | ND | 67.99 | 148.55 |
| | 102011 | 3.00 | 1410.00 | 6744 | ND | ND | 290.99 | 501.79 |
| | N | 6 | 6 | 6 | 6 | 6 | 6 | 6 |
| | Mean | 3.167 | 1346.833 | 8059.0 | ND | ND | 335.791 | 372.185 |
| | SD | 0.753 | 712.663 | 4080.4 | ND | ND | 170.018 | 122.521 |
| | Min | 2.00 | 101.00 | 1632 | ND | ND | 67.99 | 148.55 |
| | Median | 3.00 | 1555.00 | 7787.6 | ND | ND | 324.48 | 377.75 |
| | Max | 4.00 | 2010.00 | 12571 | ND | ND | 523.80 | 501.79 |
| | CV % | 23.8 | 52.9 | 50.6 | ND | ND | 50.6 | 32.9 |
| 10 | 102001 | 4.00 | 1450.00 | 12003 | 12287 | 2.32 | 434.33 | 316.79 |
| | 102003 | 2.00 | 1730.00 | 11557 | 11959 | 3.36 | 412.65 | 401.43 |
| | 102005 | 3.00 | 1430.00 | 9531 | 9759 | 2.33 | 369.52 | 372.87 |
| | 102006 | 3.00 | 1320.00 | 14024 | 15111 | 7.19 | 459.84 | 266.63 |
| | 102009 | 4.00 | 245.00 | 6077 | 6805 | 10.70 | 130.57 | 135.41 |
| | 102011 | 4.00 | 1470.00 | 10643 | 10834 | 1.77 | 406.57 | 342.45 |
| | N | 6 | 6 | 6 | 6 | 6 | 6 | 6 |
| | Mean | 3.333 | 1274.167 | 10639.1 | 11126.0 | 4.612 | 368.913 | 305.929 |
| | SD | 0.816 | 522.038 | 2690.6 | 2776.6 | 3.572 | 120.571 | 95.577 |
| | Min | 2.00 | 245.00 | 6077 | 6805 | 1.77 | 130.57 | 135.41 |
| | Median | 3.50 | 1440.00 | 11099.6 | 11396.7 | 2.85 | 409.61 | 329.62 |
| | Max | 4.00 | 1730.00 | 14024 | 15111 | 10.70 | 459.84 | 401.43 |
| | CV % | 24.5 | 41.0 | 25.3 | 25.0 | 77.5 | 32.7 | 31.2 |

| Dosing | Subject | Accum | t½ | AUC0-tau | Vz/F | CLss/F | Cmin | Tlast |
|---|---|---|---|---|---|---|---|---|
| Day | Number | Index | (h) | (h*ng/mL) | (L) | (L/h) | (ng/mL) | (h) |
| 1 | 102001 | ND | 7.07 | 8831 | ND | ND | ND | 24.00 |
| | 102003 | ND | 5.60 | 12137 | ND | ND | ND | 24.00 |
| | 102005 | ND | 4.61 | 12571 | ND | ND | ND | 24.00 |
| | 102006 | ND | 9.17 | 6439 | ND | ND | ND | 24.00 |
| | 102009 | ND | 17.12 | 1632 | ND | ND | ND | 24.00 |
| | 102011 | ND | 8.29 | 6744 | ND | ND | ND | 24.00 |
| | N | 6 | 6 | 6 | 6 | 6 | 6 | 6 |
| | Mean | ND | 8.643 | 8059.0 | ND | ND | ND | 24.000 |
| | SD | ND | 4.477 | 4080.4 | ND | ND | ND | 0.000 |
| | Min | ND | 4.61 | 1632 | ND | ND | ND | 24.00 |
| | Median | ND | 7.7 | 7787.58 | ND | ND | ND | 24.00 |
| | Max | ND | 17.12 | 12571 | ND | ND | ND | 24.00 |
| | CV % | ND | 51.8 | 50.6 | ND | ND | ND | 0.0 |
| 10 | 102001 | 1.44 | 13.99 | 10424 | 387.37 | 19.19 | 74.10 | 60.00 |
| | 102003 | 1.78 | 20.21 | 9904 | 588.86 | 20.19 | 73.50 | 72.00 |
| | 102005 | 1.34 | 12.03 | 8868 | 391.51 | 22.55 | 52.20 | 48.00 |
| | 102006 | 2.88 | 39.04 | 11036 | 1020.57 | 18.12 | 93.90 | 96.00 |
| | 102009 | 2.25 | 28.35 | 3134 | 2610.40 | 63.82 | 68.20 | 96.00 |
| | 102011 | 1.22 | 9.63 | 9758 | 284.65 | 20.50 | 77.70 | 48.00 |
| | N | 6 | 6 | 6 | 6 | 6 | 6 | 6 |
| | Mean | 1.818 | 20.542 | 8853.9 | 880.559 | 27.396 | 73.267 | 70.000 |
| | SD | 0.642 | 11.292 | 2893.7 | 887.079 | 17.907 | 13.532 | 22.018 |
| | Min | 1.22 | 9.63 | 3134 | 284.65 | 18.12 | 52.20 | 48.00 |
| | Median | 1.61 | 17.1 | 9830.64 | 490.18 | 20.35 | 73.80 | 66.00 |
| | Max | 2.88 | 39.04 | 11036 | 2610.40 | 63.82 | 93.90 | 96.00 |
| | CV % | 35.3 | 55.0 | 32.7 | 100.7 | 65.4 | 18.5 | 31.5 |

Part 2 of the study was dosed with food at 200 mg daily (QD) or 100 mg twice daily (BID). It was apparent that both QD and BD dosing resulted in mean accumulation indexes of approximately 2±1. Following BID dosing, C_{avg} doubled between days 1 and 10 but mean Cₘₐₓ increased at less than 2-fold. Following QD dosing, neither Cₘₐₓ nor C_{avg} changed between Days 1 and 10. Similarly AUC₀₋ₜₐᵤ did not change appreciably. The fluctuation between Cₘₐₓ and Cₘᵢₙ was higher following QD dosing compared to BID dosing. The predicted accumulation following multiple dosing (QD dosing: 1.4 and 2.5 times and BID (2 and 4.5), based upon kel was achieved in Part 2 (but at the lower end of the predicted range). The Cₘᵢₙ concentrations upon 200 mg QD dosing were 52-94 ng/mL (mean 73 ng/mL), whereas those for 100 mg BID were 117-195 ng/mL (mean 146 ng/mL).

Disclosed herein are the following concepts:
1. A method of treating or preventing a symptom associated with an endocrine disturbance in a subject in need thereof, the method comprising administering twice daily to the subject an effective amount of a pharmaceutical composition comprising a compound of formula (I):

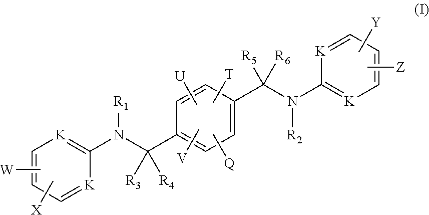

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R, and one or more pharmaceutically acceptable excipients.
2. Use of a pharmaceutical composition comprising an effective amount of a compound of formula (I):

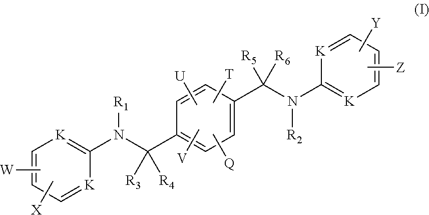

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients, in the manufacture of a medicament for treating or preventing a symptom associated with an endocrine disturbance, wherein the medicament is for twice daily administration.
3. Use of an effective amount of a pharmaceutical composition comprising a compound of formula (I):

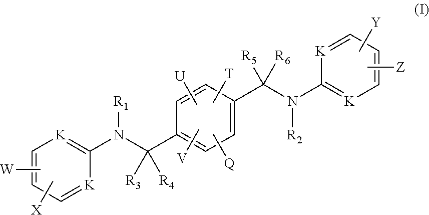

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients, for the treatment or prevention of a symptom associated with an endocrine disturbance in a subject.
4. A compound of formula (I) or a pharmaceutical composition comprising formula (I):
comprising an effective amount of a compound of formula (I):

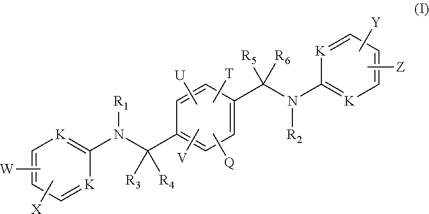

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients, for use in the treatment or prevention of a symptom associated with an endocrine disturbance in a subject.
5. The method, use, compound or pharmaceutical composition according to any one of items 1 to 4, wherein the compound or the pharmaceutical composition is administered to the subject twice a day.
6. The method, use, compound or pharmaceutical composition according to any one of items 1 to 5, wherein the compound or the pharmaceutical composition is administered with a consumable.
7. The method, use, compound or pharmaceutical composition according to item 6, wherein the consumable is a high-fat consumable.
8. The method, use, compound or pharmaceutical composition according to item 7, wherein the high-fat consumable a high-fat food or meal, or an edible fat or oil.
9. The method, use, compound or pharmaceutical composition according to any one of items 1 to 8, wherein the compound or the pharmaceutical composition is administered to the subject in an amount of between about 50 mg and about 400 mg per dose.
10. The method, use, compound or pharmaceutical composition according to item 9, wherein the amount is between about 60 mg and about 350 mg per dose.
11. The method, use, compound or pharmaceutical composition according to item 10, wherein the amount is between about 70 mg and about 300 mg per dose.
12. The method, use, compound or pharmaceutical composition according to item 11, wherein the amount is between about 80 mg and about 250 mg per dose.
13. The method, use, compound or pharmaceutical composition according to item 12, wherein the amount is between about 90 mg and about 200 mg.
14. The method, use, compound or pharmaceutical composition according to item 13, wherein the amount is about 100 mg per dose.
15. A method of treating or preventing a symptom associated with an endocrine disturbance in a subject, the method comprising administering a pharmaceutical composition comprising an effective amount of a compound of formula (I):

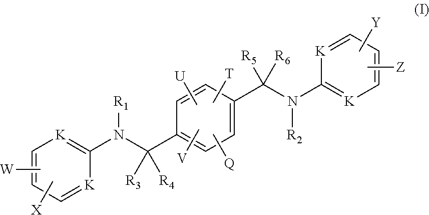

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients, to an individual in combination with, or at the same time as, consumption of a high-fat consumable.
16. A method of treating or preventing hot flashes in a subject, the method comprising administering a compound of formula (I) or a pharmaceutical composition comprising formula (I): comprising an effective amount of a compound of formula (I):

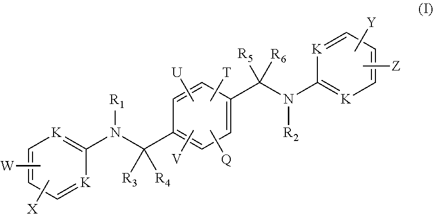

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients, in an amount and at an interval of 100 mg of compound of formula (I) twice every 24 hours.
17. The method of item 16, wherein 100 mg of the compound is administered once every 12 hours.
18. The method, use, compound or pharmaceutical composition according to any one of items 1 to 17, wherein the symptom is a hot flash.
19. The method, use, compound or pharmaceutical composition according to any one of items 1 to 18, wherein the pharmaceutical composition comprises a compound of formula (I) in an amount sufficient to provide a C_{trough} of about 150 to about 350 ng/mL when the composition is orally administered to the subject.
20. The method, use, compound or pharmaceutical composition according to any one of items 1 to 18, wherein the pharmaceutical composition comprises a compound of formula (I) in an amount sufficient to provide a mean C_{trough} of about 230 ng/mL when the composition is orally administered to the subject.
21. The method, use, compound or pharmaceutical composition according to any one of items 1 to 18, wherein the pharmaceutical composition comprises a compound of formula (I) in an amount sufficient to provide a C_{trough} of greater than 130 ng/mL when the composition is orally administered to a subject.
22. The method, use, compound or pharmaceutical composition according to any one of items 1 to 21, wherein Q, T, U, and V are independently selected from H or methyl.
23. The method, use, compound or pharmaceutical composition according to item 22, wherein Q, T, U, and V are H.
24. The method, use, compound or pharmaceutical composition according to any one of items 1 to 23, wherein W, X, Y and Z are independently selected from H, R, F, Cl, OH, OR or CO₂H.
25. The method, use, compound or pharmaceutical composition according to item 24, wherein W, X, Y and Z are independently selected from H, R, F, Cl, OR or CO₂H.
26. The method, use, compound or pharmaceutical composition according to item 25, wherein W, X, Y and Z are be independently selected from H, R, F, Cl or OR.
27. The method, use, compound or pharmaceutical composition according to item 26, wherein W, X, Y and Z are independently selected from H.
28. The method, use, compound or pharmaceutical composition according to item 27, wherein W, X, Y and Z are H.
29. The method, use, compound or pharmaceutical composition according to any one of items 1 to 28, wherein R is a straight chain alkyl group.
30. The method, use, compound or pharmaceutical composition according to any one of items 1 to 28, wherein R is a branched alkyl group.
31. The method, use, compound or pharmaceutical composition according to item 29 or item 30, wherein R is a C₁-C₁₀ alkyl group.
32. The method, use, compound or pharmaceutical composition according to item 31, wherein R is a C₁-C₅ alkyl group.
33. The method, use, compound or pharmaceutical composition according to item 32, wherein R is a C₁-C₃ alkyl group.
34. The method, use, compound or pharmaceutical composition according to item 33, wherein R is methyl or ethyl.
35. The method, use, compound or pharmaceutical composition according to any one of items 1 to 34, wherein R₁ and R₂ are independently selected from H.
36. The method, use, compound or pharmaceutical composition according to any one of items 1 to 35, wherein R₃, R₄, R₅ and R₆ are independently selected from H.
37. The method, use, compound or pharmaceutical composition according to any one of items 1 to 21, wherein the compound is a compound of formula (Ia):

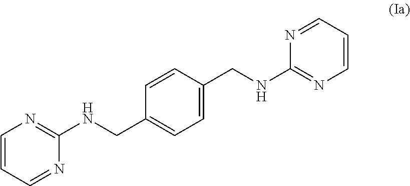 [SMILES: c1cnc(NCc2ccc(CNc3ncccn3)cc2)nc1]

or a pharmaceutically acceptable salt or prodrug thereof.
38. A pharmaceutical composition comprising an effective amount of a compound of formula (I):

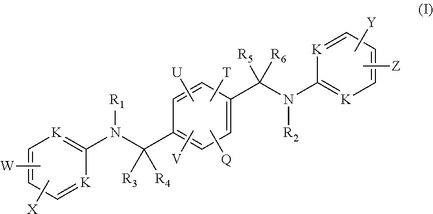

or a pharmaceutically acceptable salt or prodrug thereof, wherein:

each K is independently N or CH, wherein at least two K's are N;

Q, T, U, and V are independently selected from H or R;

W, X, Y and Z are independently selected from H, R, F, Cl, Br, I, OH, OR or CO₂H;

R is an alkyl group;

R₁ and R₂ are independently selected from H or R, and

R₃, R₄, R₅ and R₆ are independently selected from H or R,

and one or more pharmaceutically acceptable excipients.
39. A pharmaceutical composition according to item 38, wherein Q, T, U, and V are independently selected from H or methyl.
40. A pharmaceutical composition according to item 38 or item 39, wherein Q, T, U, and V are H.
41. A pharmaceutical composition according to any one of items 38 to 40, wherein W, X, Y and Z are independently selected from H, R, F, Cl, OH, OR or CO₂H.
42. A pharmaceutical composition according to item 41, wherein W, X, Y and Z are independently selected from H, R, F, Cl, OR or CO₂H.
43. A pharmaceutical composition according to item 42, wherein W, X, Y and Z are be independently selected from H, R, F, Cl or OR.
44. A pharmaceutical composition according to item 43, wherein W, X, Y and Z are independently selected from H.
45. A pharmaceutical composition according to item 44, wherein W, X, Y and Z are H.
46. A pharmaceutical composition according to any one of items 38 to 45, wherein R is a straight chain alkyl group.
47. A pharmaceutical composition according to any one of items 38 to 45, wherein R is a branched alkyl group.
48. A pharmaceutical composition according to item 46 or item 47, wherein R is a C₁-C₁₀ alkyl group.
49. A pharmaceutical composition according to item 48, wherein R is a C₁-C₅ alkyl group.
50. A pharmaceutical composition according to item 49, wherein R is a C₁-C₃ alkyl group.
51. A pharmaceutical composition according to item 50, wherein R is methyl or ethyl.
52. A pharmaceutical composition according to any one of items 38 to 51, wherein R₁ and R₂ are independently selected from H.
53. A pharmaceutical composition according to any one of items 38 to 52, wherein R₃, R₄, R₅ and R₆ are independently selected from H.
54. A pharmaceutical composition according to item 38, wherein the compound is a compound of formula (Ia):

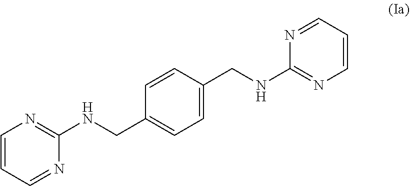 [SMILES: c1cnc(NCc2ccc(CNc3ncccn3)cc2)nc1]

or a pharmaceutically acceptable salt or prodrug thereof.
55. A pharmaceutical composition according to any one of items 38 to 54, wherein the effective amount is between about 50 mg and about 400 mg per dose.
56. A pharmaceutical composition according to item 55, wherein the effective amount is between about 60 mg and about 350 mg per dose.
57. A pharmaceutical composition according to item 56, wherein the effective amount is between about 70 mg and about 300 mg per dose.
58. A pharmaceutical composition according to item 57, wherein the effective amount is between about 80 mg and about 250 mg per dose.
59. A pharmaceutical composition according to item 58, wherein the effective amount is between about 90 mg and about 200 mg per dose.
60. A pharmaceutical composition according to item 59, wherein the effective amount is about 100 mg per dose.
61. A pharmaceutical composition according to any one of items 38 to 60, wherein the pharmaceutical composition is formulated for oral administration.
62. A pharmaceutical composition according to any one of items 38 to 61, wherein the pharmaceutical composition is suitable for, or adapted for, administration twice a day.
63. A pharmaceutical composition according to any one of items 38 to 62, wherein the the pharmaceutical composition is suitable for, or adapted for, administration with a consumable.
64. A pharmaceutical composition according to item 63, wherein the consumable is a high-fat consumable.
65. A pharmaceutical composition according to item 64, wherein the consumable is a high-fat food or meal, or an edible fat or oil.
66. A kit for treating or preventing a symptom associated with an endocrine disturbance, the kit comprising at least two dosage units,

wherein the kit is adapted to allow access to each dosage unit at different times,

wherein both the first and second dosage unit each separately comprise compound (I), (Ia) or a pharmaceutical composition comprising compound (I) or (Ia) according to any one of the preceding items.
67. The kit according to item 66, wherein the first and second dosage unit each separately comprise between about 50 mg and about 400 mg of compound (I) or (Ia).
68. The kit according to item 67, wherein the first and second dosage unit each separately comprise about 100 mg of compound (I) or (Ia).
69. A kit for treating or preventing a symptom associated with an endocrine disturbance, the kit comprising:

a first dosage unit comprising compound (I), (Ia) or a pharmaceutical composition comprising compound (I) or (Ia) according to any one of items 38 to 65;

a second dosage unit comprising compound (I), (Ia) or a pharmaceutical composition comprising compound (I) or (Ia) according to any one of items 38 to 65;

indicia distinguishing the first and second dosage units from each other;

instructions for coordinating the administration of each of the first and second dosage units as a treatment or prevention regimen whereby the first and second dosage units are for separate administration within a 24 hour period; and

a container which incorporates the indicia, the instructions and a plurality of first and second analgesic dosage units.
70. The kit according to item 69, wherein the first and second dosage unit each separately comprise between about 50 mg and about 400 mg of compound (I) or (Ia).
71. The kit according to item 70, wherein the first and second dosage unit each separately comprise about 100 mg of compound (I) or (Ia).
72. The kit according to any one of items 66 to 71, wherein the pharmaceutical composition is a tablet.
73. The kit according to any one of items 66 to 72, wherein the pharmaceutical composition is provided in a blister card.
74. The kit according to any one of items 66 to 73, wherein the pharmaceutical composition is contained within the kit with instructions to take the pharmaceutical composition with a high fat consumable.
75. The kit according to any one of items 66 to 74, wherein the pharmaceutical composition is contained within the kit with instructions to take the pharmaceutical composition twice a day.
76. The kit according to any one of items 66 to 75, wherein the symptom is hot flashes.
77. A pharmaceutical composition according to any one of items 38 to 65, wherein the pharmaceutical composition comprises a compound of formula (I) in an amount sufficient to provide a C_{trough} of about 150 to about 350 ng/mL when the composition is orally administered to a subject.
78. The pharmaceutical composition according to item 77, wherein the C_{trough} is about 150 to about 340 ng/mL.
79. The pharmaceutical composition according to item 78, wherein the C_{trough} is about 150 to about 330 ng/mL.
80. The pharmaceutical composition according to item 79, wherein the C_{trough} is about 150 to about 320 ng/mL.
81. The pharmaceutical composition according to item 80, wherein the C_{trough} is about 150 to about 310 ng/mL.
82. The pharmaceutical composition according to item 81, wherein the C_{trough} is about 150 to about 300 ng/mL.
83. The pharmaceutical composition according to any one of items 38 to 65, wherein the composition comprises a compound of formula (I) in an amount sufficient to provide a mean C_{trough} of about 230 ng/mL when the composition is orally administered to a subject.
84. The pharmaceutical composition according to any one of items 38 to 65, wherein the composition comprises a compound of formula (I) in an amount sufficient to provide a C_{trough} of greater than 130 ng/mL when the composition is orally administered to a subject.

1. A method of treating or preventing a symptom associated with an endocrine disturbance in a subject in need thereof, the method comprising administering twice daily to the subject an effective amount of a compound of formula (I): 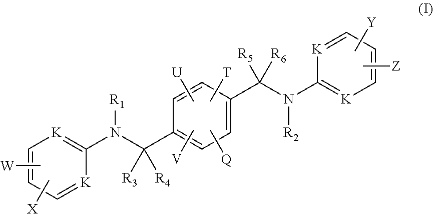
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R.

2. Use of a compound of formula (I): 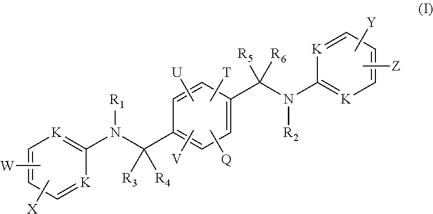
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
in the manufacture of a medicament for treating or preventing a symptom associated with an endocrine disturbance, wherein the medicament is formulated for twice daily administration of the compound or pharmaceutically acceptable salt or prodrug thereof.

3. Use of an effective amount of a compound of formula (I): 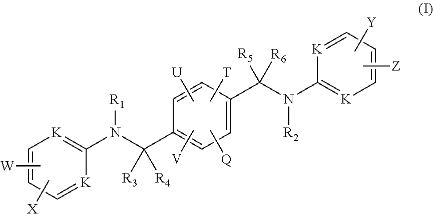
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
for the treatment or prevention of a symptom associated with an endocrine disturbance in a subject, wherein the effective amount is for twice daily administration of the pharmaceutical composition.

4. A compound of formula (I) 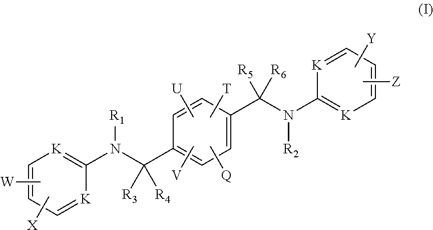
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
and one or more pharmaceutically acceptable excipients, for use in the treatment or prevention of a symptom associated with an endocrine disturbance in a subject, wherein the treatment or prevention comprises twice daily administration of the compound.

5. A pharmaceutical composition comprising a compound of formula (I): 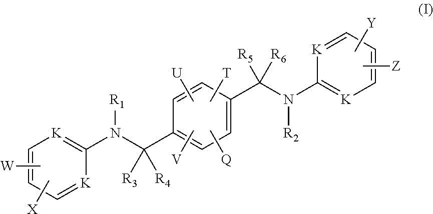
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
and one or more pharmaceutically acceptable excipients, for use in the treatment or prevention of a symptom associated with an endocrine disturbance in a subject, wherein the treatment or prevention comprises twice daily administration of the pharmaceutical composition.

6. The method, use, compound or pharmaceutical composition according to any one of claims 1 to 5, wherein the compound or the pharmaceutical composition is administered with a consumable.

7. The method, use, compound or pharmaceutical composition according to claim 6, wherein the consumable is a high-fat consumable.

8. The method, use, compound or pharmaceutical composition according to claim 7, wherein the high-fat consumable a high-fat food or meal, or an edible fat or oil.

9. The method, use, compound or pharmaceutical composition according to any one of claims 1 to 8, wherein the compound or the pharmaceutical composition is administered to the subject in an amount of between about 50 mg and about 400 mg per dose.

10. The method, use, compound or pharmaceutical composition according to claim 9, wherein the amount is between about 60 mg and about 350 mg per dose.

11. The method, use, compound or pharmaceutical composition according to claim 10, wherein the amount is about 100 mg per dose.

12. The method, use, compound or pharmaceutical composition according to any one of claims 1 to 11, wherein the symptom is a hot flash.

13. A method of treating or preventing hot flashes in a subject, the method comprising administering an effective amount a compound of formula (I) or an effective amount a pharmaceutical composition comprising formula (I): comprising a compound of formula (I): 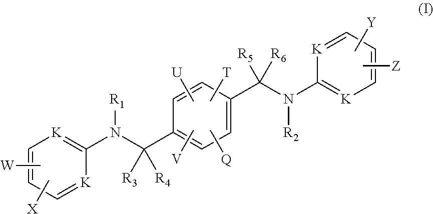
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
and one or more pharmaceutically acceptable excipients, in an amount and at an interval of 100 mg of compound of formula (I) twice every 24 hours.

14. The method of claim 12, wherein 100 mg of the compound is administered once every 12 hours.

15. The method, use, compound or pharmaceutical composition according to any one of claims 1 to 14, wherein Q, T, U, and V are independently selected from H or methyl.

16. The method, use, compound or pharmaceutical composition according to claim 15, wherein Q, T, U, and V are H.

17. The method, use, compound or pharmaceutical composition according to any one of claims 1 to 16, wherein W, X, Y and Z are independently selected from H, R, F, Cl, OH, OR or CO₂H.

18. The method, use, compound or pharmaceutical composition according to claim 17, wherein W, X, Y and Z are independently selected from H, R, F, Cl, OR or CO₂H.

19. The method, use, compound or pharmaceutical composition according to claim 18, wherein W, X, Y and Z are independently selected from H, R, F, Cl or OR.

20. The method, use, compound or pharmaceutical composition according to claim 19, wherein each of W, X, Y and Z is H.

21. The method, use, compound or pharmaceutical composition according to any one of claims 1 to 20, wherein R₁ and R₂ are independently selected from H.

22. The method, use, compound or pharmaceutical composition according to any one of claims 1 to 21, wherein R₃, R₄, R₅ and R₆ are independently selected from H.

23. The method, use, compound or pharmaceutical composition according to any one of claims 1 to 22, wherein the compound is a compound of formula (Ia): 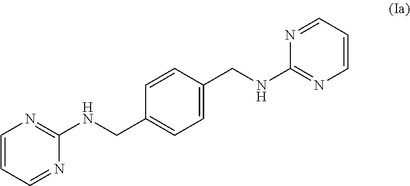 [SMILES: c1cnc(NCc2ccc(CNc3ncccn3)cc2)nc1]
or a pharmaceutically acceptable salt or prodrug thereof.

24. A unit dosage form comprising a compound of formula (I): 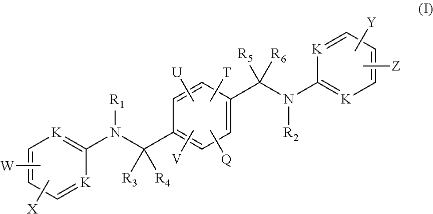
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
and one or more pharmaceutically acceptable excipients,
wherein the unit dosage form comprises the compound of formula (I) or pharmaceutically acceptable salt or prodrug thereof in an amount sufficient for twice daily administration to a subject in need thereof.

25. The unit dosage form according to claim 24, comprising from about 60 mg to about 400 mg of the compound of formula (I) or pharmaceutically acceptable salt or prodrug thereof.

26. The unit dosage form according to claim 24 or 25, comprising about 100 mg of the compound of formula (I) or pharmaceutically acceptable salt or prodrug thereof.

27. The unit dosage form according to any one of claims 24 to 26, formulated for oral administration.

28. A kit comprising in separate compartments,
a first unit dosage form according to any one of claims 24 to 27;
a second unit dosage form according to any one of claims 24 to 27,
wherein the kit is adapted to allow access to each compartment at a different time.

29. The kit of claim 28, wherein the first and second unit dosage form comprise the same amount of the compound of formula (I) or a pharmaceutically acceptable salt or prodrug thereof.

30. The kit of claim 28 or 29, further comprising indicia distinguishing the first and second unit dosage forms from each other.

31. The kit of any one of claims 28 to 30, comprising a plurality of first unit dosage forms and a plurality of second unit dosage forms, each unit dosage forms within either plurality contained in a separate container adapted for to allow access at different times.

32. The kit according to any one of claims 28 to 31, wherein the first and second unit dosage form is provided in the form of a tablet.

33. The kit according to any one of claims 28 to 32, wherein the containers are provided in the form of a blister pack.

34. The kit of any one of claims 28 to 31, further comprising instructions for coordinating the administration of the first and second dosage units as part of a treatment or prevention regimen comprising separate administration of a first unit dosage form and a second unit dosage form within a 24 hour period.

35. The kit according to claim 34, wherein the instructions coordinate the subject to take the first and second unit dosage forms with a high fat consumable.

36. A method of treating or preventing a symptom associated with an endocrine disturbance in a subject in need thereof, the method comprising administering to the subject an effective amount of a compound of formula (I): 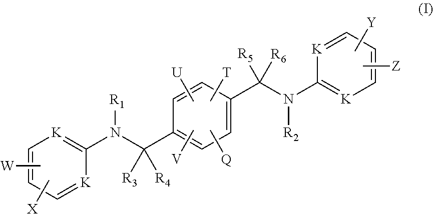
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
in combination with, or at the same time as, consumption of a high-fat consumable.

37. The method of claim 36, wherein the compound is administered once every 24 hours.

38. The method of claim 36, wherein the compound is administered twice every 24 hours.

39. The method of any one of claims 36 to 38, wherein the high-fat consumable a high-fat food or meal, or an edible fat or oil.

40. The method according to any one of claims 36 to 39, wherein the compound is administered to the subject in an amount of between about 50 mg and about 400 mg per dose.

41. The method according to any one of claims 36 to 40, wherein the amount is between about 60 mg and about 350 mg per dose.

42. The method according to any one of claims 36 to 41, wherein the amount is about 100 mg per dose.

43. The method according to any one of claims 36 to 42, wherein the symptom is a hot flash.

44. The method according to any one of claims 36 to 43, wherein Q, T, U, and V are independently selected from H or methyl.

45. The method according to claim 44, wherein Q, T, U, and V are H.

46. The method according to any one of claims 36 to 45, wherein W, X, Y and Z are independently selected from H, R, F, Cl, OH, OR or CO₂H.

47. The method according to claim 46, wherein W, X, Y and Z are independently selected from H, R, F, Cl, OR or CO₂H.

48. The method according to claim 47, wherein W, X, Y and Z are independently selected from H, R, F, Cl or OR.

49. The method according to claim 48, wherein each of W, X, Y and Z is H.

50. The method according to any one of claims 36 to 49, wherein R₁ and R₂ are independently selected from H.

51. The method according to any one of claims 36 to 50, wherein R₃, R₄, R₅ and R₆ are independently selected from H.

52. The method according to any one of claims 36 to 51, wherein the compound is a compound of formula (Ia): 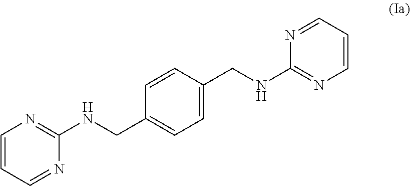 [SMILES: c1cnc(NCc2ccc(CNc3ncccn3)cc2)nc1]
or a pharmaceutically acceptable salt or prodrug thereof.

53. Use of an effective amount of a compound of formula (I): 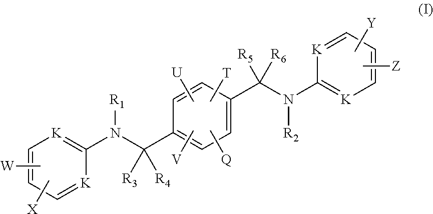
or a pharmaceutically acceptable salt or prodrug thereof, wherein:
each K is independently N or CH, wherein at least two K's are N;
Q, T, U, and V are independently selected from H or R;
W, X, Y and Z are independently selected from H, R, F, Cl, Br, I, OH, OR or CO₂H;
R is an alkyl group;
R₁ and R₂ are independently selected from H or R, and
R₃, R₄, R₅ and R₆ are independently selected from H or R,
in the manufacture of a medicament for the treatment or prevention of a symptom associated with an endocrine disturbance in a subject, wherein the medicament is for administration with the consumption of a high-fat consumable.

54. The use according to claim 53, wherein the compound is a compound of formula (Ia): 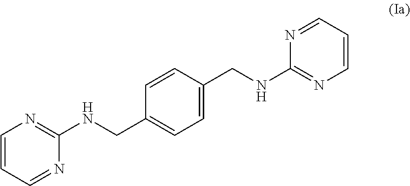 [SMILES: c1cnc(NCc2ccc(CNc3ncccn3)cc2)nc1]
or a pharmaceutically acceptable salt or prodrug thereof.
